SEQUENCE LISTING (138 sequences)

SEQ ID NO: 1 - protein, 124 aa, Artificial Sequence
  synthetic anti-PcrV (V antigen of Pseudomonas aeruginosa type III secretion system (TTSS)) antibody Vh (VH1) V-region
EIQLVQSGAEVKKPGASVKVSCKASGYTFTDHAISWVRQAPGQGLEWMGWISPYSGNPNYAQSLQGRVSLTTDRSTRTAYMELRSLKSDDTAVYYCARNRGDIYYDFTYAFDIWGQGTMVTVSS

SEQ ID NO: 2 - protein, 107 aa, Artificial Sequence
  synthetic anti-PcrV (V antigen of Pseudomonas aeruginosa type III secretion system (TTSS)) antibody VkI V-region
DIQMTQSPSSVSASVGDRVTITCRASQGISSWLAWYQQKPGRAPKLLIYAASSLQSGVPSRFSGSGSGTGFTLTISSLQPEDVATYYCQQFWSTPYTFGQGTKLEIK

SEQ ID NO: 3 - protein, 124 aa, Artificial Sequence
  synthetic anti-PcrV (V antigen of Pseudomonas aeruginosa type III secretion system (TTSS)) antibody Vh V-region
QVQLVESGGGVVQPGGSLRLSCAASGFTFSTAGMHWVRQAPGKGLEWVAVIWYNGKEISYADSVKGRFTVSRDNPKNTLYLQMSSLRTEDTAVYYCARNRGDIYYDFTYAMDIWGQGTMVTVSS

SEQ ID NO: 4 - protein, 107 aa, Artificial Sequence
  synthetic anti-PcrV (V antigen of Pseudomonas aeruginosa type III secretion system (TTSS)) antibody Vh (VH1) V-region
DIQMTQSPSSLSASVGDRVTITCRASQSISRWVAWYQQRPGKAPNLLIYDASSLKSGVPSRFSGSGSGTEFTLTISSLQPEDIATYYCQQFWSTPYTFGQGTKLEIK

SEQ ID NO: 5 - protein, 124 aa, Artificial Sequence
  synthetic anti-PcrV (V antigen of Pseudomonas aeruginosa type III secretion system (TTSS)) antibody VkI V-region
QVQLVESGGGVVQPGRSLRLSCTASGFSFSSYGMHWVRQAPGKGLEWVAVIWYNGKEISYADSVKGRFTVSRDNPKNTLYLQMSSLRTEDTAVYYCARNRGDIYYDFTYAMDIWGQGTMVTVSS

SEQ ID NO: 6 - protein, 107 aa, Artificial Sequence
  synthetic anti-PcrV (V antigen of Pseudomonas aeruginosa type III secretion system (TTSS)) antibody VkI V-region
AIQLTQSPSFLSASVGDRVTITCRASQGISTYLAWYQQKPGKAPKLLIYAASSLQSGVPSRFSGSGSGTDFTLTISSLQPEDVATYYCQQFWSTPYTFGQGTKLEIK

SEQ ID NO: 7 - protein, 124 aa, Artificial Sequence
  synthetic anti-PcrV (V antigen of Pseudomonas aeruginosa type III secretion system (TTSS)) antibody Vh V-region
QVQLVESGGGLVQPGRSLRLSCVGSGFTFSSYGIHWVRQAPGKGLEWVAVIWYNGKEISYADSVKGRFTVSRDNLKNTLYLQMSSLRTEDTAVYYCARNRGDIYYDFTYAMDIWGQGTMVTVSS

SEQ ID NO: 8 - protein, 107 aa, Artificial Sequence
  synthetic anti-PcrV (V antigen of Pseudomonas aeruginosa type III secretion system (TTSS)) antibody VkI V-region
DIQMTQSPSFLSASVGDRVTITCRASQGISTYLAWYQQKRGKAPKLLISAASSLQSGVPSRFSGSVSGTDFTLTISSLQSEDFAVYYCQQFWSTPYTFGQGTKLEIK

SEQ ID NO: 9 - protein, 124 aa, Artificial Sequence
  synthetic anti-PcrV (V antigen of Pseudomonas aeruginosa type III secretion system (TTSS)) antibody Vh V-region
QVQLVESGGGLVQPGRSLRLSCVGSGFTFSSYGIHWVRQAPGKGLEWVAVIWYNGKEISYADSVKGRFTVSRDNPKNTLYLQMSSLRTEDTAVYYCARNRGDIYYDFTYAMDIWGQGTMVTVSS

SEQ ID NO: 10 - protein, 107 aa, Artificial Sequence
  synthetic anti-PcrV (V antigen of Pseudomonas aeruginosa type III secretion system (TTSS)) antibody VkI V-region
DIQLTQSPSFLSASVGDRVTITCRASQGISTYLAWYQQKPGKAPKLLIYDASALQSGVPSRFSGSGSGTEFTLTISSLQPEDVATYYCQQFWSTPYTFGQGTKLEIK

SEQ ID NO: 11 - protein, 124 aa, Artificial Sequence
  synthetic anti-PcrV (V antigen of Pseudomonas aeruginosa type III secretion system (TTSS)) antibody Vh V-region
EVQLVESGGGVVQPGGSLRLSCAASGFTFSTAGMHWVRQAPGKGLEWVAVIWYNGKEISYADSVKGRFTVFRDNPKNTLYLQMSSLRTEDTAVYYCARNRGDIYYDFTYAMDIWGQGTMVTVSS

SEQ ID NO: 12 - protein, 107 aa, Artificial Sequence; identical to SEQ ID NO: 4
  synthetic anti-PcrV (V antigen of Pseudomonas aeruginosa type III secretion system (TTSS)) antibody VkI V-region

SEQ ID NO: 13 - protein, 124 aa, Artificial Sequence
  synthetic anti-PcrV (V antigen of Pseudomonas aeruginosa type III secretion system (TTSS)) antibody Vh V-region
QVQLVESGGGVVQPGRSLRLSCAASGFTFSSYPLHWVRQAPGKGLEWVSFISYDGSEKYYASSVKGRFTISRDNSENTLYLQMNSLRPEDTAVYYCARNRGDIYYDFTYAMDIWGQGTMVTVSS

SEQ ID NO: 14 - protein, 107 aa, Artificial Sequence; identical to SEQ ID NO: 10
  synthetic anti-PcrV (V antigen of Pseudomonas aeruginosa type III secretion system (TTSS)) antibody Vk V-region

SEQ ID NO: 15 - protein, 124 aa, Artificial Sequence
  synthetic anti-PcrV (V antigen of Pseudomonas aeruginosa type III secretion system (TTSS)) antibody Vh V-region
EVQLVESGGGVVQPGRSLRLSCTASGFSFSSYGMHWVRQAPGKGLEWVAVIWYDGRNKYYADSVKGRFTISRDNSKNTLYLQMNSLRAEDTAVYYCARNRGDIYYDFTYAMDIWGQGTMVTVSS

SEQ ID NO: 16 - protein, 108 aa, Artificial Sequence
  synthetic anti-PcrV (V antigen of Pseudomonas aeruginosa type III secretion system (TTSS)) antibody VkIII V-region
EIVLTQFPGTLSLSPGERATLSCRASQNVGSAYLAWYQQKPGQAPRLLIYGASRRAPGIPDRFSGSGSGTDFTLTINRLEPEDFAVYYCQQFWSTPYTFGQGTKLEIK

SEQ ID NO: 17 - protein, 124 aa, Artificial Sequence
  synthetic anti-PcrV (V antigen of Pseudomonas aeruginosa type III secretion system (TTSS)) antibody Vh V-region
EVQLVESGGGVVQPGRSLRLSCAASGFTFSSYGMHWVRQAPGKGLEWVAVIWYDGYNKDYADSVKGRFTISRDNSKNTLYLQINSLRAEDTAVYYCARNRGDIYYDFTYAMDIWGQGTMVTVSS

SEQ ID NO: 18 - protein, 107 aa, Artificial Sequence
  synthetic anti-PcrV (V antigen of Pseudomonas aeruginosa type III secretion system (TTSS)) antibody VkIII V-region
EIVMTQSPATLSVSPGERATLSCRASQSVSSNLAWYQQKPGQAPRLLIYGASTRATGIPARFSGSGSGTEFTLTISSLQSEDFAVYYCQQFWSTPYTFGQGTKLEIK

SEQ ID NO: 19 - protein, 124 aa, Artificial Sequence
  synthetic anti-PcrV (V antigen of Pseudomonas aeruginosa type III secretion system (TTSS)) antibody Vh V-region
EVQLVESGGGVVQPGRSLRLSCAASGFTFSSYPLHWVRQAPGKGLEWVSFISYDGSEKYYASSVKGRFTISRDNSENTLYLQMNSLRPEDTAVYYCARNRGDIYYDFTYAMDIWGQGTMVTVSS

SEQ ID NO: 20 - protein, 107 aa, Artificial Sequence
  synthetic anti-PcrV (V antigen of Pseudomonas aeruginosa type III secretion system (TTSS)) antibody VkIII V-region
EIVMTQSPATLSVSPGERATLSCRASQSVSSNLAWYQQKPGQAPRLLFYAASTRATGIPARFSGSGSGTEFTLTISSLQSEDFAVYYCQQFWSTPYTFGQGTKLEIK

SEQ ID NO: 21 - protein, 124 aa, Artificial Sequence
  synthetic anti-PcrV (V antigen of Pseudomonas aeruginosa type III secretion system (TTSS)) antibody Vh V-region
EVQLVESGGGLVQPGRSLRLSCVGSGFTFSSYGIHWVRQAPGKGLEWVANIWYDGSSESYIDSVKGRFTVSRDDSRNTVYLQMNSLRPEDTAVYYCARNRGDIYYDFTYAMDIWGQGTMVTVSS

SEQ ID NO: 22 - protein, 107 aa, Artificial Sequence; identical to SEQ ID NO: 18
  synthetic anti-PcrV (V antigen of Pseudomonas aeruginosa type III secretion system (TTSS)) antibody VkIII V-region

SEQ ID NO: 23 - protein, 124 aa, Artificial Sequence
  synthetic anti-PcrV (V antigen of Pseudomonas aeruginosa type III secretion system (TTSS)) antibody VH V-region
EVQLVESGGGVVQPGRSLRLSCAASGFTFSNYPMHWVRQAPGKGLEWVAVISYDGSEKWYADSVKGRFTISRDNSKNTLYLEMNSLRPEDTAVYYCARNRGDIYYDFTYAMDQWGQGTTVTVSS

SEQ ID NO: 24 - protein, 107 aa, Artificial Sequence
  synthetic anti-PcrV (V antigen of Pseudomonas aeruginosa type III secretion system (TTSS)) antibody VK V-region
DIQLTQSPSTLSASVGDSVTITCRASEGVDRWLAWYQQKPGRAPKLLIYDASTLQSGVPSRFSGSGSGTEFSLTISSLQPDDVATYYCQHFWGTPYTFGQGTKLEIK

SEQ ID NO: 25 - protein, 124 aa, Artificial Sequence
  synthetic anti-PcrV (V antigen of Pseudomonas aeruginosa type III secretion system (TTSS)) antibody VH V-region
EVQLVESGGGVVQPGRSLRLSCAASGFTFSNYPMHWVRQAPGKGLEWVAVISYDGSEKWYADSVKGRFTISRDNSKNTLYLEMNSLRPEDTAVYYCARNRGDIYYDFTYAMDSWGQGTTVTVSS

SEQ ID NO: 26 - protein, 124 aa, Artificial Sequence
  synthetic anti-PcrV (V antigen of Pseudomonas aeruginosa type III secretion system (TTSS)) antibody VH V-region
EVQLVESGGGVVQPGRSLRLSCAASGFTFSNYPMHWVRQAPGKGLEWVAVISYDGSEKWYADSVKGRFTISRDNSKNTLYLEMNSLRPEDTAVYYCARNRGDIYYDFTYAMDIWGQGTTVTVSS

SEQ ID NO: 27 - protein, 124 aa, Artificial Sequence; identical to SEQ ID NO: 19
  synthetic anti-PcrV (V antigen of Pseudomonas aeruginosa type III secretion system (TTSS)) antibody Vh V-region

SEQ ID NO: 28 - protein, 104 aa, Artificial Sequence
  synthetic anti-PcrV (V antigen of Pseudomonas aeruginosa type III secretion system (TTSS)) antibody Vl V-region
QSALTQPASVSGSPGQSITISCTGTSSDYVSWYQQHPGKAPKLIIYDVTNRPSGVPDRFSGSKSGNTASLTISGLQAEDEADYYCQHFWSTPYTFGGGTKLTVL

SEQ ID NO: 29 - protein, 124 aa, Artificial Sequence; identical to SEQ ID NO: 19
  synthetic anti-PcrV (V antigen of Pseudomonas aeruginosa type III secretion system (TTSS)) antibody Vh V-region

SEQ ID NO: 30 - protein, 106 aa, Artificial Sequence
  synthetic anti-PcrV (V antigen of Pseudomonas aeruginosa type III secretion system (TTSS)) antibody Vl V-region
SSELTQDPAVSVALGQTVRITCQGDSLRSYYASWYQQKPGQAPVLVIYGKNNRPSGIPDRFSGSSSGNTASLTITGAQAEDEADYYCQHFWSTPYTFGGGTKLTVL

SEQ ID NO: 31 - protein, 5 aa, Artificial Sequence
  synthetic anti-PcrV (V antigen of Pseudomonas aeruginosa type III secretion system (TTSS)) antibody CDR3
FWGTP

SEQ ID NO: 32 - protein, 106 aa, Artificial Sequence
  synthetic anti-PcrV (V antigen of Pseudomonas aeruginosa type III secretion system (TTSS)) antibody Vl V-region
SSELTQDPAVSVALGQTVTITCQGDSLRSLYASWYQQKPGQAPVLVLYSKNSRPSGIPDRFSGSSSGNTASLTITGARAEDEADYYCQHFWSTPYTFGGGTKLTVL

SEQ ID NO: 33 - protein, 9 aa, Artificial Sequence
  synthetic anti-PcrV (V antigen of Pseudomonas aeruginosa type III secretion system (TTSS)) antibody V-L region CDR3
QXFWGTPYT

SEQ ID NO: 34 - protein, 109 aa, Artificial Sequence
  synthetic anti-PcrV (V antigen of Pseudomonas aeruginosa type III secretion system (TTSS)) antibody Vl V-region
QSVLTQPPSASGSPGQSVTISCTGTSSDVGAYNYVSWYQQYPGKVPKLIIYEVTKRPSGVPDRFSGSKSGNTASLTVSGLRAEDEADYYCQHFWSTPYTFGGGTKLTVL

SEQ ID NO: 35 - protein, 124 aa, Artificial Sequence
  synthetic anti-PcrV (V antigen of Pseudomonas aeruginosa type III secretion system (TTSS)) antibody VH V-region
EVQLVESGGGVVQPGRSLRLSCAASGFTFSNYPMHWVRQAPGKGLEWVAVISYDGSEKWYADSVKGRFTISRDNSKNTLYLEMNSLRPEDTAVYYCARNRGDIYYDFTYAMDYWGQGTTVTVSS

SEQ ID NO: 36 - protein, 107 aa, Artificial Sequence
  synthetic anti-PcrV (V antigen of Pseudomonas aeruginosa type III secretion system (TTSS)) antibody VK V-region
DIQLTQSPSTLSASVGDSVTITCRASEGVDRWLAWYQQKPGRAPKLLIYDASTLQSGVPSRFSGSGSGTEFSLTISSLQPDDVATYYCQHFWSTPYTFGQGTKLEIK

SEQ ID NO: 37 - protein, 107 aa, Artificial Sequence
  synthetic anti-PcrV (V antigen of Pseudomonas aeruginosa type III secretion system (TTSS)) antibody VkI V-region
DIQMTQSPSSLSASVGDRVTITCRASQSISRWVAWYQQRPGKAPNLLIYDASSLKSGVPSRFSGSGSGTEFTLTISSLQPEDIATYYCQQFWGTPYTFGQGTKLEIK

SEQ ID NO: 38 - protein, 11 aa, Artificial Sequence
  synthetic anti-PcrV (V antigen of Pseudomonas aeruginosa type III secretion system (TTSS)) antibody V-H region CDR3
NRGDIYYDFTY

SEQ ID NO: 39 - protein, 15 aa, Artificial Sequence
  synthetic anti-PcrV (V antigen of Pseudomonas aeruginosa type III secretion system (TTSS)) antibody V-H region CDR3
NRGDIYYDFTYAXDX

SEQ ID NO: 40 - protein, 15 aa, Artificial Sequence
  synthetic anti-PcrV (V antigen of Pseudomonas aeruginosa type III secretion system (TTSS)) antibody V-H region CDR3
NRGDIYYDFTYAMDX

SEQ ID NO: 41 - protein, 11 aa, Artificial Sequence
  synthetic anti-PcrV (V antigen of Pseudomonas aeruginosa type III secretion system (TTSS)) antibody V-H region FR4 region
WGQGTSVTVSS

SEQ ID NO: 42 - protein, 5 aa, Artificial Sequence
  synthetic anti-PcrV (V antigen of Pseudomonas aeruginosa type III secretion system (TTSS)) antibody V-L region CDR3
FWXTP

SEQ ID NO: 43 - protein, 11 aa, Artificial Sequence
  synthetic anti-PcrV (V antigen of Pseudomonas aeruginosa type III secretion system (TTSS)) antibody V-H region FR4
WGQGTXVTVSS

SEQ ID NO: 44 - protein, 9 aa, Artificial Sequence
  synthetic anti-PcrV (V antigen of Pseudomonas aeruginosa type III secretion system (TTSS)) antibody light chain CDR3
QXFWXTPYT

SEQ ID NO: 45 - protein, 10 aa, Artificial Sequence
  synthetic anti-PcrV (V antigen of Pseudomonas aeruginosa type III secretion system (TTSS)) antibody V-L region FR4
FGQGTKLEIK

SEQ ID NO: 46 - protein, 10 aa, Artificial Sequence
  synthetic anti-PcrV (V antigen of Pseudomonas aeruginosa type III secretion system (TTSS)) antibody V-L region FR4
FGGGTKLTVL

SEQ ID NO: 47 - protein, 17 aa, Artificial Sequence
  synthetic anti-PcrV (V antigen of Pseudomonas aeruginosa type III secretion system (TTSS)) antibody heavy chain region CDR2
XIXYBGXXXXYXXSVKG

SEQ ID NO: 48 - protein, 5 aa, Artificial Sequence
  synthetic anti-PcrV (V antigen of Pseudomonas aeruginosa type III secretion system (TTSS)) antibody V-H region V-segment CDR1
TAGMH

SEQ ID NO: 49 - protein, 5 aa, Artificial Sequence
  synthetic anti-PcrV (V antigen of Pseudomonas aeruginosa type III secretion system (TTSS)) antibody V-H region V-segment CDR1
SYGIH

SEQ ID NO: 50 - protein, 5 aa, Artificial Sequence
  synthetic anti-PcrV (V antigen of Pseudomonas aeruginosa type III secretion system (TTSS)) antibody V-H region V-segment CDR1
SYGMH

SEQ ID NO: 51 - protein, 5 aa, Artificial Sequence
  synthetic anti-PcrV (V antigen of Pseudomonas aeruginosa type III secretion system (TTSS)) antibody V-H region V-segment CDR1
SYPLH

SEQ ID NO: 52 - protein, 5 aa, Artificial Sequence
  synthetic anti-PcrV (V antigen of Pseudomonas aeruginosa type III secretion system (TTSS)) antibody V-H region V-segment CDR1
NYPMH

SEQ ID NO: 53 - protein, 17 aa, Artificial Sequence
  synthetic anti-PcrV (V antigen of Pseudomonas aeruginosa type III secretion system (TTSS)) antibody V-H region V-segment CDR2
VIWYNGKEISYADSVKG

SEQ ID NO: 54 - protein, 17 aa, Artificial Sequence
  synthetic anti-PcrV (V antigen of Pseudomonas aeruginosa type III secretion system (TTSS)) antibody V-H region V-segment CDR2
FISYDGSEKYYASSVKG

SEQ ID NO: 55 - protein, 17 aa, Artificial Sequence
  synthetic anti-PcrV (V antigen of Pseudomonas aeruginosa type III secretion system (TTSS)) antibody V-H region V-segment CDR2
VISYDGSEKWYADSVKG

SEQ ID NO: 56 - protein, 17 aa, Artificial Sequence
  synthetic anti-PcrV (V antigen of Pseudomonas aeruginosa type III secretion system (TTSS)) antibody V-H region V-segment CDR2
VIWYDGRNKYYADSVKG

SEQ ID NO: 57 - protein, 17 aa, Artificial Sequence
  synthetic anti-PcrV (V antigen of Pseudomonas aeruginosa type III secretion system (TTSS)) antibody V-H region V-segment CDR2
VIWYDGYNKDYADSVKG

SEQ ID NO: 58 - protein, 17 aa, Artificial Sequence
  synthetic anti-PcrV (V antigen of Pseudomonas aeruginosa type III secretion system (TTSS)) antibody V-H region V-segment CDR2
NIWYDGSSESYIDSVKG

SEQ ID NO: 59 - protein, 5 aa, Artificial Sequence
  synthetic anti-PcrV (V antigen of Pseudomonas aeruginosa type III secretion system (TTSS)) antibody V-H region V-segment CDR1
DHAIS

SEQ ID NO: 60 - protein, 17 aa, Artificial Sequence
  synthetic anti-PcrV (V antigen of Pseudomonas aeruginosa type III secretion system (TTSS)) antibody V-H region V-segment CDR2
WISPYSGNPNYAQSLQG

SEQ ID NO: 61 - protein, 15 aa, Artificial Sequence
  synthetic anti-PcrV (V antigen of Pseudomonas aeruginosa type III secretion system (TTSS)) antibody V-H region CDR3
NRGDIYYDFTYAFDI

SEQ ID NO: 62 - protein, 11 aa, Artificial Sequence
  synthetic anti-PcrV (V antigen of Pseudomonas aeruginosa type III secretion system (TTSS)) antibody V-L region V-segment CDR1
RASZXXXXXXA

SEQ ID NO: 63 - protein, 7 aa, Artificial Sequence
  synthetic anti-PcrV (V antigen of Pseudomonas aeruginosa type III secretion system (TTSS)) antibody V-L region V-segment CDR2
XASXLXS

SEQ ID NO: 64 - protein, 11 aa, Artificial Sequence
  synthetic anti-PcrV (V antigen of Pseudomonas aeruginosa type III secretion system (TTSS)) antibody V-L region V-segment CDR1
RASQGISTYLA

SEQ ID NO: 65 - protein, 11 aa, Artificial Sequence
  synthetic anti-PcrV (V antigen of Pseudomonas aeruginosa type III secretion system (TTSS)) antibody V-L region V-segment CDR1
RASQGISSWLA

SEQ ID NO: 66 - protein, 11 aa, Artificial Sequence
  synthetic anti-PcrV (V antigen of Pseudomonas aeruginosa type III secretion system (TTSS)) antibody V-L region V-segment CDR1
RASQSISRWVA

SEQ ID NO: 67 - protein, 11 aa, Artificial Sequence
  synthetic anti-PcrV (V antigen of Pseudomonas aeruginosa type III secretion system (TTSS)) antibody V-L region V-segment CDR1
RASEGVDRWLA

SEQ ID NO: 68 - protein, 7 aa, Artificial Sequence
  synthetic anti-PcrV (V antigen of Pseudomonas aeruginosa type III secretion system (TTSS)) antibody V-L region V-segment CDR2
AASSLQS

SEQ ID NO: 69 - protein, 7 aa, Artificial Sequence
  synthetic anti-PcrV (V antigen of Pseudomonas aeruginosa type III secretion system (TTSS)) antibody V-L region V-segment CDR2
DASSLKS

SEQ ID NO: 70 - protein, 7 aa, Artificial Sequence
  synthetic anti-PcrV (V antigen of Pseudomonas aeruginosa type III secretion system (TTSS)) antibody V-L region V-segment CDR2
DASALQS

SEQ ID NO: 71 - protein, 7 aa, Artificial Sequence
  synthetic anti-PcrV (V antigen of Pseudomonas aeruginosa type III secretion system (TTSS)) antibody V-L region V-segment CDR2
DASTLQS

SEQ ID NO: 72 - protein, 12 aa, Artificial Sequence
  synthetic anti-PcrV (V antigen of Pseudomonas aeruginosa type III secretion system (TTSS)) antibody V-L region V-segment CDR1
RASNSVGAYNLA

SEQ ID NO: 73 - protein, 11 aa, Artificial Sequence
  synthetic anti-PcrV (V antigen of Pseudomonas aeruginosa type III secretion system (TTSS)) antibody V-L region V-segment CDR1
RASQSVSSNLA

SEQ ID NO: 74 - protein, 7 aa, Artificial Sequence
  synthetic anti-PcrV (V antigen of Pseudomonas aeruginosa type III secretion system (TTSS)) antibody V-L region V-segment CDR2
XASXRAX

SEQ ID NO: 75 - protein, 11 aa, Artificial Sequence
  synthetic anti-PcrV (V antigen of Pseudomonas aeruginosa type III secretion system (TTSS)) antibody V-L region V-segment CDR1
QGDSLRSXYAS

SEQ ID NO: 76 - protein, 7 aa, Artificial Sequence
  synthetic anti-PcrV (V antigen of Pseudomonas aeruginosa type III secretion system (TTSS)) antibody V-L region V-segment CDR2
XKNXRPS

SEQ ID NO: 77 - protein, 14 aa, Artificial Sequence
  synthetic anti-PcrV (V antigen of Pseudomonas aeruginosa type III secretion system (TTSS)) antibody V-L region V-segment CDR1
TGTSSDVGAYNYVS

SEQ ID NO: 78 - protein, 9 aa, Artificial Sequence
  synthetic anti-PcrV (V antigen of Pseudomonas aeruginosa type III secretion system (TTSS)) antibody V-L region V-segment CDR1
TGTSSDYVS

SEQ ID NO: 79 - protein, 7 aa, Artificial Sequence
  synthetic anti-PcrV (V antigen of Pseudomonas aeruginosa type III secretion system (TTSS)) antibody V-L region V-segment CDR2
XVTXRPS

SEQ ID NO: 80 - protein, 113 aa, Homo sapiens
  human germ-line sequence of VH1-18 and germ-line JH3
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARAFDIWGQGTMVTVSS

SEQ ID NO: 81 - protein, 124 aa, Artificial Sequence; identical to SEQ ID NO: 1
  synthetic anti-PcrV (V antigen of Pseudomonas aeruginosa type III secretion system (TTSS)) antibody VH1 V-H region

SEQ ID NO: 82 - protein, 113 aa, Homo sapiens
  human germ-line sequence of VH3-30.3 and germ-line JH3
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARAFDIWGQGTMVTVSS

SEQ ID NO: 83 - protein, 124 aa, Artificial Sequence; identical to SEQ ID NO: 7
  synthetic anti-PcrV (V antigen of Pseudomonas aeruginosa type III secretion system (TTSS)) antibody VH3 V-H region

SEQ ID NO: 84 - protein, 124 aa, Artificial Sequence; identical to SEQ ID NO: 9
  synthetic anti-PcrV (V antigen of Pseudomonas aeruginosa type III secretion system (TTSS)) antibody VH3 V-H region

SEQ ID NO: 85 - protein, 124 aa, Artificial Sequence; identical to SEQ ID NO: 5
  synthetic anti-PcrV (V antigen of Pseudomonas aeruginosa type III secretion system (TTSS)) antibody VH3 V-H region

SEQ ID NO: 86 - protein, 124 aa, Artificial Sequence; identical to SEQ ID NO: 11
  synthetic anti-PcrV (V antigen of Pseudomonas aeruginosa type III secretion system (TTSS)) antibody VH3 V-H region

SEQ ID NO: 87 - protein, 124 aa, Artificial Sequence; identical to SEQ ID NO: 3
  synthetic anti-PcrV (V antigen of Pseudomonas aeruginosa type III secretion system (TTSS)) antibody VH3 V-H region

SEQ ID NO: 88 - protein, 124 aa, Artificial Sequence; identical to SEQ ID NO: 19
  synthetic anti-PcrV (V antigen of Pseudomonas aeruginosa type III secretion system (TTSS)) antibody VH3 V-H region

SEQ ID NO: 89 - protein, 124 aa, Artificial Sequence; identical to SEQ ID NO: 13
  synthetic anti-PcrV (V antigen of Pseudomonas aeruginosa type III secretion system (TTSS)) antibody VH3 V-H region

SEQ ID NO: 90 - protein, 124 aa, Artificial Sequence; identical to SEQ ID NO: 15
  synthetic anti-PcrV (V antigen of Pseudomonas aeruginosa type III secretion system (TTSS)) antibody VH3 V-H region

SEQ ID NO: 91 - protein, 124 aa, Artificial Sequence; identical to SEQ ID NO: 17
  synthetic anti-PcrV (V antigen of Pseudomonas aeruginosa type III secretion system (TTSS)) antibody VH3 V-H region

SEQ ID NO: 92 - protein, 124 aa, Artificial Sequence; identical to SEQ ID NO: 21
  synthetic anti-PcrV (V antigen of Pseudomonas aeruginosa type III secretion system (TTSS)) antibody VH3 V-H region

SEQ ID NO: 93 - protein, 15 aa, Homo sapiens
  human germ-line JH6
GMDVWGQGTTVTVSS

SEQ ID NO: 94 - protein, 124 aa, Artificial Sequence; identical to SEQ ID NO: 23
  synthetic anti-PcrV (V antigen of Pseudomonas aeruginosa type III secretion system (TTSS)) antibody VH3 V-H region

SEQ ID NO: 95 - protein, 124 aa, Artificial Sequence; identical to SEQ ID NO: 25
  synthetic anti-PcrV (V antigen of Pseudomonas aeruginosa type III secretion system (TTSS)) antibody VH3 V-H region

SEQ ID NO: 96 - protein, 124 aa, Artificial Sequence; identical to SEQ ID NO: 26
  synthetic anti-PcrV (V antigen of Pseudomonas aeruginosa type III secretion system (TTSS)) antibody VH3 V-H region

SEQ ID NO: 97 - protein, 124 aa, Artificial Sequence; identical to SEQ ID NO: 35
  synthetic anti-PcrV (V antigen of Pseudomonas aeruginosa type III secretion system (TTSS)) antibody VH3 V-H region

SEQ ID NO: 98 - protein, 107 aa, Homo sapiens; identical to SEQ ID NO: 24
  human germ-line sequence of VkappaI L12 and germ-line Jkappa2

SEQ ID NO: 99 - protein, 107 aa, Artificial Sequence; identical to SEQ ID NO: 2
  synthetic anti-PcrV (V antigen of Pseudomonas aeruginosa type III secretion system (TTSS)) antibody Vkappa V-L region

SEQ ID NO: 100 - protein, 107 aa, Artificial Sequence; identical to SEQ ID NO: 8
  synthetic anti-PcrV (V antigen of Pseudomonas aeruginosa type III secretion system (TTSS)) antibody Vkappa V-L region

SEQ ID NO: 101 - protein, 107 aa, Artificial Sequence; identical to SEQ ID NO: 8
  synthetic anti-PcrV (V antigen of Pseudomonas aeruginosa type III secretion system (TTSS)) antibody Vkappa V-L region

SEQ ID NO: 102 - protein, 107 aa, Artificial Sequence; identical to SEQ ID NO: 10
  synthetic anti-PcrV (V antigen of Pseudomonas aeruginosa type III secretion system (TTSS)) antibody Vkappa V-L region

SEQ ID NO: 103 - protein, 107 aa, Artificial Sequence; identical to SEQ ID NO: 6
  synthetic anti-PcrV (V antigen of Pseudomonas aeruginosa type III secretion system (TTSS)) antibody Vkappa V-L region

SEQ ID NO: 104 - protein, 107 aa, Artificial Sequence; identical to SEQ ID NO: 4
  synthetic anti-PcrV (V antigen of Pseudomonas aeruginosa type III secretion system (TTSS)) antibody Vkappa V-L region

SEQ ID NO: 105 - protein, 107 aa, Artificial Sequence; identical to SEQ ID NO: 37
  synthetic anti-PcrV (V antigen of Pseudomonas aeruginosa type III secretion system (TTSS)) antibody Vkappa V-L region

SEQ ID NO: 106 - protein, 107 aa, Artificial Sequence; identical to SEQ ID NO: 36
  synthetic anti-PcrV (V antigen of Pseudomonas aeruginosa type III secretion system (TTSS)) antibody Vkappa V-L region

SEQ ID NO: 107 - protein, 107 aa, Homo sapiens
  human germ-line sequence of VkappaIII L2 and germ-line Jkappa2
EIVMTQSPATLSVSPGERATLSCRASQSVSSNLAWYQQKPGQAPRLLIYGASTRATGIPARFSGSGSGTEFTLTISSLQSEDFAVYYCQQYNNWPYTFGQGTKLEIK

SEQ ID NO: 108 - protein, 108 aa, Artificial Sequence
  synthetic anti-PcrV (V antigen of Pseudomonas aeruginosa type III secretion system (TTSS)) antibody Vkappa V-L region
EIVLTQFPGTLSLSPGERATLSCRASNSVGAYNLAWYQQKPGQAPRLLIYGASRRAPGIPDRFSGSGSGTDFTLTINRLEPEDFAVYYCQQFWSTPYTFGQGTKLEIK

SEQ ID NO: 109 - protein, 107 aa, Artificial Sequence; identical to SEQ ID NO: 18
  synthetic anti-PcrV (V antigen of Pseudomonas aeruginosa type III secretion system (TTSS)) antibody Vkappa V-L region

SEQ ID NO: 110 - protein, 107 aa, Artificial Sequence; identical to SEQ ID NO: 20
  synthetic anti-PcrV (V antigen of Pseudomonas aeruginosa type III secretion system (TTSS)) antibody Vkappa V-L region

SEQ ID NO: 111 - protein, 108 aa, Homo sapiens
  human germ-line sequence of VL3 3l and germ-line Jl2
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 112 - protein, 106 aa, Artificial Sequence; identical to SEQ ID NO: 30
  synthetic anti-PcrV (V antigen of Pseudomonas aeruginosa type III secretion system (TTSS)) antibody Vkappa V-L region

SEQ ID NO: 113 - protein, 106 aa, Artificial Sequence; identical to SEQ ID NO: 32
  synthetic anti-PcrV (V antigen of Pseudomonas aeruginosa type III secretion system (TTSS)) antibody Vkappa V-L region

SEQ ID NO: 114 - protein, 111 aa, Homo sapiens
  human germ-line sequence of VL2 2c and germ-line Jl2
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNFVVFGGGTKLTVL

SEQ ID NO: 115 - protein, 109 aa, Artificial Sequence
  synthetic anti-PcrV (V antigen of Pseudomonas aeruginosa type III secretion system (TTSS)) antibody Vkappa V-L region
QSVLTQPPSASGSPGQSVTISCTGTSSDVGAYNYVSWYQQYPGKVPKLIIYEVTKRPSGVPDRFSGSKSGNTASLTVSGRQAEDEADYYCQHFWSTPYTFGGGTKLTVL

SEQ ID NO: 116 - protein, 104 aa, Artificial Sequence; identical to SEQ ID NO: 28
  synthetic anti-PcrV (V antigen of Pseudomonas aeruginosa type III secretion system (TTSS)) antibody Vkappa V-L region

SEQ ID NO: 117 - protein, 15 aa, Artificial Sequence
  synthetic anti-PcrV (V antigen of Pseudomonas aeruginosa type III secretion system (TTSS)) antibody heavy chain CDR3
NRGDIYYDFTYAXDX

SEQ ID NO: 118 - protein, 11 aa, Artificial Sequence
  synthetic anti-PcrV (V antigen of Pseudomonas aeruginosa type III secretion system (TTSS)) antibody FR4 sequence provided by human germ-line JH3 segment
WGQGTMVTVSS

SEQ ID NO: 119 - protein, 11 aa, Artificial Sequence
  synthetic anti-PcrV (V antigen of Pseudomonas aeruginosa type III secretion system (TTSS)) antibody FR4 sequence provided by human germ-line JH6 segment
WGQGTTVTVSS

SEQ ID NO: 120 - protein, 26 aa, Artificial Sequence
  synthetic anti-PcrV (V antigen of Pseudomonas aeruginosa type III secretion system (TTSS)) antibody combination of CDRH3 and FR4
NRGDIYYDFTYAFDIWGQGTMVTVSS

SEQ ID NO: 121 - protein, 26 aa, Artificial Sequence
  synthetic anti-PcrV (V antigen of Pseudomonas aeruginosa type III secretion system (TTSS)) antibody combination of CDRH3 and FR4
NRGDIYYDFTYAMDIWGQGTMVTVSS

SEQ ID NO: 122 - protein, 26 aa, Artificial Sequence
  synthetic anti-PcrV (V antigen of Pseudomonas aeruginosa type III secretion system (TTSS)) antibody combination of CDRH3 and FR4
NRGDIYYDFTYAMDIWGQGTTVTVSS

SEQ ID NO: 123 - protein, 15 aa, Artificial Sequence
  synthetic anti-PcrV (V antigen of Pseudomonas aeruginosa type III secretion system (TTSS)) antibody V-H region V-segment CDR3
NRGDIYYDFTYAMDI

SEQ ID NO: 124 - protein, 15 aa, Artificial Sequence
  synthetic anti-PcrV (V antigen of Pseudomonas aeruginosa type III secretion system (TTSS)) antibody V-L region V-segment CDR2
NRGDIYYDFTYAXDX

SEQ ID NO: 125 - protein, 19 aa, Artificial Sequence
  synthetic anti-PcrV (V antigen of Pseudomonas aeruginosa type III secretion system (TTSS)) antibody combination of kappa CDRL3 and FR4
QQFWSTPYTFGQGTKLEIK

SEQ ID NO: 126 - protein, 19 aa, Artificial Sequence
  synthetic anti-PcrV (V antigen of Pseudomonas aeruginosa type III secretion system (TTSS)) antibody combination of kappa CDRL3 and FR4
QHFWGTPYTFGQGTKLEIK

SEQ ID NO: 127 - protein, 19 aa, Artificial Sequence
  synthetic anti-PcrV (V antigen of Pseudomonas aeruginosa type III secretion system (TTSS)) antibody combination of lambda CDRL3 and FR4
QHFWSTPYTFGGGTKLTVL

SEQ ID NO: 128 - protein, 11 aa, Artificial Sequence
  synthetic anti-PcrV (V antigen of Pseudomonas aeruginosa type III secretion system (TTSS)) antibody V-L region V-segment CDR1
QGDSLRSYYAS

SEQ ID NO: 129 - protein, 7 aa, Artificial Sequence
  synthetic anti-PcrV (V antigen of Pseudomonas aeruginosa type III secretion system (TTSS)) antibody V-L region V-segment CDR2
GKNNRPS

SEQ ID NO: 130 - protein, 7 aa, Artificial Sequence
  synthetic anti-PcrV (V antigen of Pseudomonas aeruginosa type III secretion system (TTSS)) antibody V-L region V-segment CDR2
EVTKRPS

SEQ ID NO: 131 - protein, 7 aa, Artificial Sequence
  synthetic anti-PcrV (V antigen of Pseudomonas aeruginosa type III secretion system (TTSS)) antibody V-L region V-segment CDR2
DVTNRPS

SEQ ID NO: 132 - protein, 9 aa, Artificial Sequence
  synthetic anti-PcrV (V antigen of Pseudomonas aeruginosa type III secretion system (TTSS)) antibody V-L region V-segment CDR3
QQFWSTPYT

SEQ ID NO: 133 - protein, 9 aa, Artificial Sequence
  synthetic anti-PcrV (V antigen of Pseudomonas aeruginosa type III secretion system (TTSS)) antibody V-L region V-segment CDR3
QHFWGTPYT

SEQ ID NO: 134 - protein, 9 aa, Artificial Sequence
  synthetic anti-PcrV (V antigen of Pseudomonas aeruginosa type III secretion system (TTSS)) antibody V-L region V-segment CDR3
QHFWSTPYT

SEQ ID NO: 135 - protein, 18 aa, Artificial Sequence
  synthetic anti-PcrV (V antigen of Pseudomonas aeruginosa type III secretion system (TTSS)) antibody combination of V-L region CDR3 and FR4
QFWSTPYTFGQGTKLEIK

SEQ ID NO: 136 - protein, 19 aa, Artificial Sequence
  synthetic anti-PcrV (V antigen of Pseudomonas aeruginosa type III secretion system (TTSS)) antibody combination of V-L region CDR3 and FR4
QHFWGTPYTFGQGTKLEIK

SEQ ID NO: 137 - protein, 19 aa, Artificial Sequence
  synthetic anti-PcrV (V antigen of Pseudomonas aeruginosa type III secretion system (TTSS)) antibody combination of V-L region CDR3 and FR4
QHFWSTPYTFGGGTKLTVL

SEQ ID NO: 138 - protein, 12 aa, Artificial Sequence
  synthetic anti-PcrV (V antigen of Pseudomonas aeruginosa type III secretion system (TTSS)) antibody combination of Vkappa CDRL3 and FR4
YTFGQGTKLEIK

(19) United States
(12) Patent Application Publication
(10) Pub. No.: US 2010/0272736 A1
(43) Pub. Date: Oct. 28, 2010

(54) COMBINATION ANTIBIOTIC AND ANTIBODY THERAPY FOR THE TREATMENT OF PSEUDOMONAS AERUGINOSA INFECTION

(75) Inventors: Mark Baer, South San Francisco, CA (US); Christopher R. Bebbington, South San Francisco, CA (US); Geoffrey T. Yarranton, South San Francisco, CA (US); Susan Lynch, San Francisco, CA (US); Yuanlin Song, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: KaloBios Pharmaceuticals, Inc., South San Francisco, CA (US); The Regents of the University of California, Oakland, CA (US)

(21) Appl. No.: 12/700,599

(22) Filed: Feb. 4, 2010

Related U.S. Application Data
(60) Provisional application No. 61/149,957, filed on Feb. 4, 2009.

(51) Int. Cl. A61K 39/40; A61P 31/04

(52) U.S. Cl. 424/170.1; 424/178.1

(57) ABSTRACT

The present invention provides improved pharmaceutical compositions and methods of treating or preventing development of bacteremia associated with Pseudomonas aeruginosa infections, where the method comprises administering an antibiotic and an anti-PcrV antibody.

### CROSS-REFERENCE TO RELATED APPLICATIONS

This application claims benefit of U.S. provisional application No. 61/149,957, filed Feb. 4, 2009, which application is herein incorporated by reference.

### BACKGROUND OF THE INVENTION

Pseudomonas aeruginosa (P. aeruginosa) is an opportunistic pathogen that rarely causes disease in healthy people, but is a significant problem for critically ill or immunocompromised individuals. Infection is a major problem in individuals who have cystic fibrosis (CF), where P. aeruginosa is a causative agent in the progressive loss of lung function resulting from recurrent and chronic respiratory tract infections with the bacterium. Others at risk from P. aeruginosa infection include patients on mechanical ventilators, neutropenic cancer patients, and burn patients.

One of the mechanisms by which P. aeruginosa produces cytotoxins is the type III secretion system (TTSS). The TTSS is an important virulence factor determinant in that it inhibits host defense system. Upon activation, the TTSS apparatus translocates toxins into the cytoplasm of the host cell, resulting in cell rounding, lifting, and cell death by necrosis.

The injectosome, which is composed of over 20 proteins, spans the bacterial membrane and is responsible for secretion of these cytotoxins. It forms a needle-like projection from the bacterial cell surface. PcrV resides at the tip of this needle complex. This protein, also known as the V antigen of the P. aeruginosa TTSS, is important to the functional intoxication of host cell cytoplasm, as evidenced by the observation that PcrV-null mutants are incapable of cytotoxin secretion. One approach to disabling the TTSS virulence system of P. aeruginosa is through use of specific antibodies that bind proteins such as the V-antigen that are presented on the bacterial cell surface and play a role in cytotoxin secretion. Mab166 is a monoclonal antibody that specifically binds PcrV and was identified as protective against development of sepsis in an acute P. aeruginosa lung infection murine model (Frank et al., J. Infect. Dis., 186:64-73, 2002). In subsequent studies, monoclonal and polyclonal anti-PcrV antibodies have demonstrated efficacy against P. aeruginosa infections through either prophylactic or therapeutic immunization in a variety of animal models, e.g., for burn and acute airway infection (Faure et al., J. Immune Therapies and Vaccines 1:2, 2003; Neely et al., Burns 31:153-158, 2005; Sawa et al., Nature Med. 5:392-398, 1999).

Previous studies have shown Mab166 or polyclonal anti-PcrV antibody to be effective in blocking the lethal effects of P. aeruginosa when administered to rats, rabbits, and a murine model of acute infection (Faure et al, 2003, supra; Imamura, et al., Eur Respir J 29:965-968, 2007; and Frank, et al., 2002, supra). The TTSS causes macrophage oncosis (Dacheux et al., Infect. and Immun. 68:2916-2924, 2000); blocking this virulence system preserves immune cell activity promoting bacterial clearance.

Antibiotics are the current standard of care for P. aeruginosa infections. However, the efficacy of conventional antibiotic treatments to combat infection is rapidly diminishing. Thus, there is an urgent need to generate novel therapeutics that replace existing treatments or enhance their effectiveness. The present invention addresses this need.

### BRIEF SUMMARY OF THE INVENTION

The current invention is based on the discovery that administration of a combination of an antibiotic and an anti-PcrV antibody can be used to effectively treat a patient infected with P. aeruginosa.

In one aspect, the invention provides a method of treating bacteremia, or preventing the development of bacteremia, in a subject infected with an antibiotic-resistant strain of P. aeruginosa, the method comprising administering a therapeutically effective amount of a combination of an anti-PcrV antibody that is an antagonist of the P. aeruginosa TTSS; and the antibiotic. In some embodiment, the patient is infected with an antibiotic-resistant strain that demonstrates resistance to the antibiotic in an in vitro assay. In some embodiments, the patient is infected with a P. aeruginosa strain that has in vivo resistance to the antibiotic. In some embodiments the antibiotic is being dosed at its maximum tolerated dose (MTD). In some embodiments the antibody is dosed by injection and the antibiotic is dosed by inhalation. In some embodiments, the antibiotic is an aminoglycoside such as tobramycin. In some embodiments the antibiotic is the monobactam aztreonam lysine. In some embodiments the antibiotic induces expression of the TTSS, e.g. tetracycline. In some embodiments, the antibiotic is piperacillin. It is understood in the art that for administration, piperacillin typically includes tazobactam. In some embodiments, the method may comprise a step of determining the level of bacteria in the blood. This can be accomplished by many methods known in the art, e.g., by culturing the blood to grow bacteria that may be present, or by using an assay such as an immunoassay to detect one or more P. aeruginosa antigens, or by using an assay such as an amplification reaction to detect P. aeruginosa nucleic acids.

The invention also provides a method of effectively enhancing the sensitivity of an antibiotic-resistant P. aeruginosa strain to the antibiotic when treating a subject infected with the strain, the method comprising administering to the subject the antibiotic and an anti-PcrV antibody that is an antagonist of the P. aeruginosa TTSS.

The methods of the invention can be used to treat any subject having a P. aeruginosa infection. Often, the subject has cystic fibrosis, is on a mechanical ventilator, is a neutropenic cancer patient, or is a burn patient. The subject need not be human, but can also be an animal, such as a bovine, equine, ovine, porcine, canine, feline, primate, or any other animal.

The methods of the invention for the combination treatment of a subject infected with P. aeruginosa can employ any antibody that neutralizes PcrV, but in some embodiments use an anti-PcrV antibody that competes with Mab166 for binding to PcrV.

The invention also provides pharmaceutical compositions comprising the anti-PcrV antibody formulated for use in combination with an antibiotic to treat a subject as described herein. Thus, in some embodiments, the invention provides a pharmaceutical composition for use in treating or preventing bacteremia in a subject infected with an antibiotic-resistant strain of P. aeruginosa and undergoing treatment with the antibiotic, the pharmaceutical composition comprising an amount of an anti-PcrV antibody that treats or prevents baceterima in the antibiotic-treated patient. The antibiotic may be an aminoglycoside, such as tobramycin. In some embodiments, the antibiotic induces the Type III secretion system. In some embodiments, the pharmaceutical composition comprising the anti-PcrV antibody is formulated such that the level of bacteria in the blood is reduced when the composition is administered of the patient. In some embodiments, the P. aeruginosa strain is resistant to the antibiotic in vivo.

The invention also provides a pharmaceutical composition comprising a therapeutically effective amount of anti-PcrV antibody that is an antagonist of the Type III secretion system for use with an antibiotic in treating or preventing an antibiotic-resistant P. aeruginosa infection in a subject. In some embodiments, the antibiotic is ineffective when administered at its maximum tolerated dose in the absence of administration of the antibody. In some embodiments, the subject does not have increased toxicity to the antibiotic dose (when administered with the pharmaceutical composition comprising the anti-PcrV antibody) compared to the maximum tolerated dose of the antibiotic when the antibiotic is administered alone to the subject. In some embodiments the antibiotic induces the expression of the Type III secretion system. In some embodiments, the antibiotic is a tetracycline, minocycline, eoxycycline, demeclocycline or oxytetracycline.

The invention additionally provides a pharmaceutical composition comprising a therapeutically effective amount of anti-PcrV antibody that is an antagonist of the Type III secretion system for use with an antibiotic in treating a subject with a P. aeruginosa lung infection, wherein the pharmaceutical composition is formulated for administering intravenously, intramuscularly, or subcutaneously, and the antibiotic is formulated for administration into the lung. In some embodiments, the antibiotic used in conjunction with the anti-PcrV antibody pharmaceutical composition is formulated to be administered by insufflation. In some embodiments, the antibiotic is tobramycin. In other embodiments, the antibiotic is aztreonam. In some embodiments, the amount of the anti-PcrV antibody and the amount of the antibiotic prevents or treats P. aeruginosa bacteremia.

The invention additionally provides a pharmaceutical composition comprising a therapeutically effective amount of anti-PcrV antibody that is an antagonist of the Type III secretion system for use with an antibiotic in treating or preventing bacteremia in a subject with a P. aeruginosa infection in a tissue other than the lung, wherein the pharmaceutical composition is formulated for intravenous administration. In some embodiments, the tissue is bladder or urinary tract tissue.

The invention additionally provides a pharmaceutical composition comprising a therapeutically effective amount of anti-PcrV antibody that is an antagonist of the Type III secretion system for use with an antibiotic to enhance the sensitivity of an antibiotic-resistant strain in a subject infected with the strain of P. aeruginosa. In some embodiments, the antibiotic is piperacillin.

A pharmaceutical composition for any of the uses described herein may be formulated for administration to a patient that has cystic fibrosis, is on a mechanical ventilator, is a neutropenic cancer patient, or is a burn patient. Further, the pharmaceutical composition may comprise an anti-PcrV antibody that is formulated for administration intravenously, intramuscularly, subcutaneously or by insufflation.

Further, in any of the uses described herein where a pharmaceutical composition is administered to a patient, the antibiotic may be formulated for intravenous, intramuscular, intradermal, or subcutaneous administration; or for insufflation.

The methods and pharmaceutical compositions can employ any anti-PcrV antibody described herein. Thus, the use of an anti-PcrV antibody in combination with an antibiotic for treatment of a patient can employ antibodies as set forth in the embodiments below.

In some embodiments, an anti-PcrV antibody for use in the methods and pharmaceutical formulations of the invention selectively binds to PcrV and comprises: a V_{L} region that comprises a CDR3 comprising FWGTP. In typical embodiments, such an antibody has a V_{L} region V-segment has at least 80% identity to a human germline V-segment. The FR4 region typically has at least 90% identity to the FR4 region of a human germline J segment.

In some embodiments, an anti-PcrV antibody for use in the methods of the invention comprises a CDR3 comprising FWGTP, a FR4 and a V-segment, wherein the FR4 comprises at least 90% identity to the FR4 region of the human JK2 germline gene segment or at least 90% identity to the JL2 germline sequence; and the V-segment comprises at least 80% identity to a human germline Vkappa I or Vkappa III sequence, or at least 80% identity to a human germline Vlambda sequence. In some embodiments the V_{L} region CDR3 has the sequence Q(Q/H)FWGTPYT. In some embodiments, the antibody further comprises a V_{H} region that comprises a CDR3 having a sequence NRGDIYYDFTY, a FR4 and a V-segment, wherein the FR4 comprises at least 90% identity to the FR4 region of the human JH3 or human JH6 segment and the V-segment comprises at least 80% identity to the human VH1-18 subclass V-segment or to the human VH3-30.3 V segment. In some embodiments, the V_{H} region comprises a CDR3 having a sequence NRGDIYYDFTYA(M/F)DX₁, wherein X₁ is I, Q, Y, or S.

In further embodiments, the invention provides an anti-PcrV antibody for use in the invention that binds to PcrV and comprises: a V_{H} region that comprises a CDR3 having a sequence NRGDIYYDFTYAMDX₁, wherein X₁ is I, Q, Y, or S; a FR4 and a V-segment, wherein the FR4 comprises at least 90% identity to the FR4 region of the human germline JH3 segment or the FR4 region of the human germine JH6 segment, and the V-segment comprises at least 80% identity to the human germline VH1-18 subclass V-segment or to the human germline VH3-30.3 subclass V segment, with the proviso that when X1 is Y, the FR4 region is not WGQGTSVTVSS.

In some embodiments, the invention provides an anti-PcrV antibody for use in the invention that binds to PcrV and comprises: a V_{H} region that comprises a CDR3 having a sequence NRGDIYYDFTYAMDX₁, wherein X₁ is I, Q, Y, or S; a FR4 and a V-segment, wherein the FR4 comprises at least 90% identity to the FR4 region of the human germline JH3 segment or the FR4 region of the human germline JH6 segment, and the V-segment comprises at least 80% identity to the human germline VH1-18 subclass V-segment or to the human germline VH3-30.3 subclass V segment, with the proviso that when X₁ is Y, the FR4 region is not WGQGTSVTVSS; and a V_{L} region that comprises a CDR3 comprising FW(S/G)TP, a FR4 and a V-segment, wherein the FR4 comprises at least 90% identity to the FR4 region of the human germline JK2 gene segment or to the FR4 region of the human germline JL2 segment; and the V-segment comprises at least 80% identity to the human germline VKI L12 sequence, or at least 80% identity to a Vkappa III sequence, or at least 80% identity to a human germline Vlambda2 2c or Vlambda3 31 segment. In some embodiments, the FR4 of the V_{H} region of an antibody for use in the invention has the sequence WGQGTX₂VTVSS, wherein X₂ is T or M.

In some embodiments, an antibody for use in the invention has a light chain CDR3 that has the sequence Q(H/Q)FW(G/S)TPYT. In some embodiments, the FR4 of the V_{L} region has the sequence FGQGTKLEIK or FGGGTKLTVL.

In some embodiments, an anti-PcrV antibody for use in the invention is one where the V_{H} region V-segment has at least 80% identity to the human germline VH3-30.3 segment and the heavy chain region CDR1 comprises the sequence X₃X₄X₅X₆H, wherein X₃ is S, T, or N; X₄ is Y or A; X₅ is A, G, or P; and X₆ is M, I, or L; and the heavy chain region CDR2 comprises the sequence X₇IX₈YX₉GX₁₀X₁₁X₁₂X₁₃Y(A/I)X₁₄SVKG, wherein X₇ is V, F, or N; X₈ is S or W; X₉ is D or N; X₁₀ is S, K, R or Y; X₁₁ is N, S, D or E; X₁₂ is K, I, or E; X13 is Y, S, D or W; and X14 is D or S. In some embodiments, the antibody has at least 90% identity to a VH3-30.3 V segment. In some embodiments, the CDR1 is TAGMH, SYGIH, SYGMH, SYPLH, or NYPMH. In some embodiments, the CDR2 is VIWYNGKEISYADSVKG, FISYDGSEKYYASSVKG, VISYDGSEKWYADSVKG, VIWYDGRNKYYADSVKG, VIWYDGYNKDYADSVKG, or NIWYDGSSESYIDSVKG. In some embodiments, the CDR1 is TAGMH, SYGIH, SYGMH, SYPLH, or NYPMH; and the CDR2 is VIWYNGKEISYADSVKG, FISYDGSEKYYASSVKG, VISYDGSEKWYADSVKG, VIWYDGRNKYYADSVKG, VIWYDGYNKDYADSVKG, or NIWYDGSSESYIDSVKG.

In some embodiments, an anti-PcrV antibody for use in the invention is one in which the V_{H} region V-segment has at least 80% identity, or at least 90% identity, to the human germline VH1-18 sub-class V-segment and the CDR1 has the sequence DHAIS and the CDR2 has the sequence WISPYSGNPNYAQSLQG.

In some embodiments, an anti-PcrV antibody for use in the invention comprises: a V_{H} region that has a CDR3 sequence NRGDIYYDFTYAFDI, a CDR1 sequence DHAIS and a CDR2 sequence WISPYSGNPNYAQSLQG.

In some embodiments, an antibody for use in the invention comprises the V_{H}-segment region of an amino acid sequence selected from the group consisting of SEQ ID NOs 1, 3, 5, 7, 9, 11, 13, 15, 17, 19, 21, 23, 25, 26, 27, 29, and 35; and the heavy chain CDR3 comprises NRGDIYYDFTYAMDX₁, wherein Xᵢ is I, Q, Y, or S; or NRGDIYYDFTYAFDI. For example, the V_{H} regions can comprise an amino acid sequence selected from the group consisting of SEQ ID NOs 1, 3, 5, 7, 9, 11, 13, 15, 17, 19, 21, 23, 25, 26, 27, 29, and 35.

In some embodiments, an anti-PcrV antibody for use in the invention is one where the V_{L} region V-segment comprises at least 80% or at least 90% identity, to a human germline Vkappa 1 L12 or Vkappa III sequence; or at least 80% or at least 90% identity to a human germline Vlambda3 31 or to a Vlambda2 2c sequence. In some embodiments, V_{L} region V-segment has at least 80% or at least 90% identity to the human germline VKI L12 segment and the CDR1 has the sequence RASX₁₅X₁₆X₁₇X₁₈X₁₉X₂₀X₂₁A, where X₁₅ is Q or E; X₁₆ is S or G; X₁₇ is I or V; X₁₈ is S or D; X₁₉ is S, R, or T; X₂₀ is W or Y; and X₂₁ is L or V; and the CDR2 has the sequence X₂₁ASX₂₂LX₂₃S, wherein X₂₁ is D or A; X₂₂ is S, A, or T; and X₂₃ is E, Q, or K. In some embodiments, the CDR1 has the sequence RASQGISTYLA, RASQGISSWLA, RASQSISRWVA, or RASEGVDRWLA; or the CDR2 has the sequence AASSLQS, DASSLKS, AASSLQS, DASALQS, or DASTLQS. In some embodiments, the CDR1 has the sequence RASQGISTYLA, RASQGISSWLA, RASQSISRWVA, or RASEGVDRWLA; and the CDR2 has the sequence AASSLQS, DASSLKS, AASSLQS, DASALQS, or DASTLQS.

In some embodiments, an anti-PcrV antibody for use in the invention is one where the V_{L} region V segment has at least 80%, or at least 90%, amino acid sequence identity to the human germline VKIII L2 sequence and the CDR1 has the sequence RASNSVGAYNLA or RASQSVSSNLA; or the CDR2 has the sequence (A/G)AS(T/R)RA(T/P). In some embodiments, CDR1 has the sequence RASNSVGAYNLA or RASQSVSSNLA; and the CDR2 has the sequence (A/G)AS(T/R)RA(T/P).

In some embodiments, an anti-PcrV antibody for use in the invention has a V_{L} region V-segment that has at least 80%, or at least 90%, amino acid sequence identity to a human germline Vlambda L3 31 segment and the CDR1 has the sequence QGDSLRS(Y/L)YAS; or the CDR2 has the sequence (G/S)KN(N/S)RPS. In some embodiments, the CDR1 has the sequence QGDSLRS(Y/L)YAS; and the CDR2 has the sequence (G/S)KN(N/S)RPS.

In some embodiments, an anti-PcrV antibody for use in the invention has a V_{L} region V-segment that has at least 80%, or at least 90%, amino acid sequence identity to a human germline Vlambda L2 2c segment and the CDR1 has the sequence TGTSSDVGAYNYVS or TGTSSDYVS; or the CDR2 has the sequence (E/D)VT(K/N)RPS. In some embodiments, the CDR1 has the sequence TGTSSDVGAYNYVS or TGTSSDYVS; and the CDR2 has the sequence (E/D)VT(K/N)RPS.

In some embodiments, an anti-PcrV antibody for use in the invention has a region that comprises the V-segment of an amino acid sequence selected from the group consisting of SEQ ID NO:2, 4, 6, 8, 10, 12, 14, 16, 18, 20, 22, 24, 28, 30, 32, 34, 36, and 37 and has a light chain CDR3 that comprises has the sequence Q(H/Q)FW(G/S)TPYT. For example the V_{L} region can comprise an amino acid sequence selected from the group consisting of SEQ ID NO:2, 4, 6, 8, 10, 12, 14, 16, 18, 20, 22, 24, 28, 30, 32, 34, 36, and 37.

In some embodiments, an anti-PcrV antibody for use in the invention comprises: a V_{H} region having an amino acid sequence selected from the group consisting of SEQ ID NOs 1, 3, 5, 7, 9, 11, 13, 15, 17, 19, 21, 23, 25, 26, 27, 29, and 35; and a V_{L} region having an amino acid sequence selected from the group consisting of SEQ ID NO:2, 4, 6, 8, 10, 12, 14, 16, 18, 20, 22, 24, 28, 30, 32, 34, 36, and 37. Thus, in some embodiments, an antibody for use in the invention comprises a V_{H} region of SEQ ID NO:1 and a V_{L} region of SEQ ID NO:2; or a V_{H} region of SEQ ID NO:3 and a V_{L} region of SEQ ID NO:4; or a V_{H} region of SEQ ID NO:5 and a V_{L} region of SEQ ID NO:6; or a V_{H} region of SEQ ID NO:7 and a V_{L} region of SEQ ID NO:8; or a V_{H} region of SEQ ID NO:11 and a V_{L} region of SEQ ID NO:12; or a V_{H} region of SEQ ID NO:9 and a V_{L} region of SEQ ID NO:10; or a V_{H} region of SEQ ID NO:13 and a V_{L} region of SEQ ID NO:10; or a V_{H} region of SEQ ID NO:13 and a V_{L} region of SEQ ID NO:4; or a V_{H} region of SEQ ID NO:13 and a V_{L} region of SEQ ID NO:37; or a V_{H} region of SEQ ID NO:21 and a V_{L} region of SEQ ID NO:18; or a V_{H} region of SEQ ID NO:17 and a V_{L} region of SEQ ID NO:18; or a V_{H} region of SEQ ID NO:26 and a V_{L} region of SEQ ID NO:24; or a V_{H} region of SEQ ID NO:25 and a V_{L} region of SEQ ID NO:24; or a V_{H} region of SEQ ID NO:23 and a V_{L} region of SEQ ID NO:24; or a V_{H} region of SEQ ID NO:35 and a V_{L} region of SEQ ID NO:36; or V_{H} region of SEQ ID NO:29 and a V_{L} region of SEQ ID NO:20; or V_{H} region of SEQ ID NO:29 and a V_{L} region of SEQ ID NO:28; or a V_{H} region of SEQ ID NO:29 and a V_{L} region of SEQ ID NO:30; or a V_{H} region of SEQ ID NO:29 and a V_{L} region of SEQ ID NO:34; or a V_{H} region of SEQ ID NO:3 and a V_{L} region of SEQ ID NO:32.

In some embodiments, an anti-PcrV antibody for use in the invention comprises a heavy chain as set forth in FIG. 8 and/or a light chain as set forth in FIG. 9; or has at least one, often at least two, and in some embodiments, at least three CDRs from one of the heavy or light chains set forth in FIG. 8 or FIG. 9, respectively. In many embodiments, the CDR1 and/or CDR2 sequence is not a germline sequence.

In some embodiments, an antibody for use in the invention is a Fab or Fab′ that has an affinity of about 10 nM or less. In some embodiments, the antibody has an affinity that is equal or better than, the affinity of a Mab166 Fab or Fab′.

The potency of an antibody for use in the invention, e.g., a Fab, in inhibiting the activity of the P. aeruginosa TTSS is typically equivalent to Mab166 Fab (within two-fold of the activity in cell-based assays). In some embodiments, the antibody is more potent than Mab166 in preventing cytotoxicity by P. aeruginosa.

In some embodiments, the anti-PcrV antibody for use in the invention competes with Mab166 for binding to PcrV.

In some embodiments, the antibody comprises a hinge region.

In other embodiments, the antibody is an IgG or an IgA.

In some embodiments, the antibody is PEGylated, e.g., di-PEGylated or mono-Pegylated.

In some embodiments, the V_{H} region or the V_{L} region, or both the V_{H} and V_{L} region amino acid sequences comprise a methionine at the N-terminus.

The antibody can be administered to the patient using any route of administration, but is often administered intravenously, intramuscularly, subcutaneously or by insufflation.

The antibiotic can also be administered to the subject using any route of administration known in the art. Typically, the antibiotic is administered intravenously, intramuscularly, or by insufflation.

### BRIEF DESCRIPTION OF THE DRAWINGS

FIG. 1. A. Body temperature of mice in control and three treatment groups (Mab166 only; antibiotic only; Mab166 and antibiotic). B. Survival curves of mice from the same groups examined in A.

FIG. 2. A. Wet/dry weight ratio of lungs from mice in control and treatment groups (Mab166; Tobramycin; Mab166 and Tobramycin) at discreet time points post-infection B. Excess lung water (ELW) in mice from control and treatment groups at discreet time points post-infection.

FIG. 3 provides data showing that Mab166/Tobramycin combination therapy protects neutrophils in lungs of infected animals. Neutrophil number per microscope field (200×) after cell cytospin and stain from bronchoalveolar lavage fluid is shown in the graph.

FIG. 4. A. Total P. aeruginosa CFU's in the lungs of mice in the control and treatment groups (Mab166; Tobramycin; Mab166 and Tobramycin), 8 and 24 hours post P. aeruginosa instillation. B. Total P. aeruginosa CFU's in the blood of mice in the control and treatment groups, 8 and 24 hours post P. aeruginosa instillation. C. Total P. aeruginosa CFU's in the spleen of mice in the control and treatment groups 8 and 24 hours post P. aeruginosa instillation.

FIG. 5. MPO concentrations in mouse plasma 8 and 24 hours post P. aeruginosa instillation in control and treatment groups (Mab166; Tobramycin; Mab166 and Tobramycin).

FIG. 6. Body temperature 4 treatment groups: control; Piperacillin; Mab166; and Mab166 and Piperacillin. Piperacillin is administered as a combination with tazobactam (a pencillinase inhibitor).

FIG. 7. Survival curves: control; Piperacillin; Mab166; and Mab166 and Piperacillin. Piperacillin is administered as a combination with tazobactam.

FIG. 8 shows sequences of V_{H} regions of anti-PcrV antibodies. CDR sequences are underlined. The VH1 sequence is aligned to human germ-line sequence VH1-18. VH3-subclass antibodies are shown aligned to human germ-line sequence VH3-30.3. J-segments are aligned to either human germ-line JH3 or JH6. The V_{H}-segments depicted in FIG. 8 correspond to the sequence up to the CDR3 sequence.

FIG. 9 shows sequences of V_{L} regions of anti-PcrV antibodies. CDR sequences are underlined. Vkappa-subclass antibodies are shown aligned to human germline sequence VKI L12. J-segments are aligned to human germ-line JK2. Vlambda-subclass antibodies are shown aligned to human germline sequence V13 31. J-segments are aligned to human germ-line JL2.

FIG. 10 provides data showing a time course of survival of mice treated with various doses of antibodies to PcrV at the time of challenge with a lethal dose of PA103. Mab166 and Fab fragments were co-instilled via the intratracheal route with 1.5×10⁶ bacteria (5 mice per group, 4 mice for Mab166 Fab groups). Control is a nonspecific Fab with no binding to PcrV or any P. aeruginosa protein. Mice were treated with antibody doses of: A) 10 μg, B) 5 μg, C) 2.5 μg, D) 1.25 μg, *P=0.01 for Fab 1A8 vs. Mab166 Fab E) 0.625 μg *P=0.002 for 1A8 vs. Mab166 Fab, F) 0.3125 μg, G) 0.16 μg, H) 0.08 μg. P values for differences between treatment groups determined by Mantel-Cox log-rank test.

FIG. 11 provides data showing a body temperature analysis of mice treated with anti-PcrV antibodies. Rectal temperatures are shown for 48 hours or until mortality. Antibody doses: A) 10 μg, B) 5 μg, C) 2.5 μg, D) 1.25 μg E) 0.625 μg F) 0.3125 μg, G) 0.16 μg, H) 0.08 μg.

FIG. 12 provides data showing clearance of P. aeruginosa from the lungs of infected mice by anti-PcrV antibodies. Mice were infected with 1.5×10⁶ cfu PA103 co-instilled with Mab166 IgG, Mab166 Fab or human Fab 1A8 at the doses shown (in μg). The graph shows cfu/lung isolated from individual mice surviving at 48 h. The number of dead mice at this time point is shown above the figure. Median cfu/lung for surviving mice in each group is shown with a bar.

### DETAILED DESCRIPTION OF THE INVENTION

### Definitions

As used herein, “synergy” refers to an effect in combination where the end result is greater than the effect obtained with the sum of each of the parts of the combination taken separately.

“Bacteremia” or “septicemia” refers to the presence of live bacteria in the bloodstream. Typically in bacteremia or septicemia there is a sufficient number such that bacteria can be cultured from a sample of blood from the patient.

A “maximum tolerated dose”, or “MTD” refers to the highest dose of a drug or treatment that does not cause unacceptable side effects.

As used herein, an “antibody” refers to a protein functionally defined as a binding protein and structurally defined as comprising an amino acid sequence that is recognized by one of skill as being derived from the framework region of an immunoglobulin-encoding gene of an animal that produces antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

The term antibody as used herein includes antibody fragments that retain binding specificity. For example, there are a number of well characterized antibody fragments. Thus, for example, pepsin digests an antibody C-terminal to the disulfide linkages in the hinge region to produce F(ab)′₂, a dimer of Fab which itself is a light chain joined to VH-CH1 (Fd) by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab with all or part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term “antibody” also includes antibody fragments produced either by the modification of whole antibodies or synthesized using recombinant DNA methodologies.

Antibodies of the invention include dimers such as V_{H}-V_{L} dimers, V_{H} dimers, or V_{L} dimers, including single chain antibodies (antibodies that exist as a single polypeptide chain), such as single chain Fv antibodies (sFv or scFv) in which a variable heavy and a variable light region are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment, such as a disulfide-stabilized Fv (dsFv). Other fragments can also be generated, including using recombinant techniques. The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). In some embodiments, antibodies include those that have been displayed on phage or generated by recombinant technology using vectors where the chains are secreted as soluble proteins, e.g., scFv, Fv, Fab, (Fab′)₂ or generated by recombinant technology using vectors where the chains are secreted as soluble proteins. Antibodies for use in the invention can also include diantibodies and miniantibodies. Further, antibodies of the invention include heavy chain dimers, such as antibodies from camelids. Since the V_{H} region of a heavy chain dimer IgG in a camelid does not have to make hydrophobic interactions with a light chain, the region in the heavy chain that normally contacts a light chain is changed to hydrophilic amino acid residues in a camelid. V_{H} domains of heavy-chain dimer IgGs are called VHH domains. Antibodies of the invention include single domain antibodies (dAbs) and nanobodies (see, e.g., Cortez-Retamozo, et al., Cancer Res. 64:2853-2857, 2004).

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation. A “V-segment” as used herein refers to the region of the V-region (heavy or light chain) that is encoded by a V gene. The V-segment of the heavy chain variable region encodes FR1-CDR1-FR2-CDR2 and FR3. For the purposes of this invention, the V-segment of the light chain variable region is defined as extending though FR3 up to CDR3.

As used herein, the term “J-segment” refers to a subsequence of the encoded variable region comprising a C-terminal portion of a CDR3 and the FR4. An endogenous J-segment is encoded by an immunoglobulin J-gene.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, for example, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl. Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “binding specificity determinant” or “BSD” as used in the context of the current invention refers to the minimum contiguous or non-contiguous amino acid sequence within a CDR region necessary for determining the binding specificity of an antibody. In the current invention, the minimum binding specificity determinants reside within a portion or the full-length of the CDR3 sequences of the heavy and light chains of the antibody.

As used herein, the terms “PcrV antagonizing antibody”, or “PcrV antibody antagonist” or “antagonist PcrV antibody, or an “anti-PcrV antibody antagonist of the Pseudomonas aeruginosa Type III secretion system (TTSS)” are used interchangeably to refer to an antibody that binds to PcrV and inhibits the TTSS. Inhibition occurs when secretion through the TTSS is at least about 10% less, for example, at least about 25%, 50%, 75% less, or totally inhibited, in comparison to secretion when not exposed to the antibody antagonist. The terms “anti-PcrV antibody” and “PcrV antibody” are used synonymously unless otherwise stated.

The term “equilibrium dissociation constant (K_{D}) refers to the dissociation rate constant (k_{d}, time⁻¹) divided by the association rate constant (kₐ, time₋₁, M⁻¹). Equilibrium dissociation constants can be measured using any known method in the art. The antibodies of the present invention are high affinity antibodies. Such antibodies have an affinity better than 500 nM, and often better than 50 nM or 10 nM. Thus, in some embodiments, the antibodies of the invention have an affinity in the range of 500 nM to 100 pM, or in the range of 50 or 25 nM to 100 pM, or in the range of 50 or 25 nM to 50 pM, or in the range of 50 nM or 25 nM to 1 pM.

As used herein, “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci. USA 91: 969, 1994).

A “humaneered” antibody in the context of this invention refers to an engineered human antibody having a binding specificity of a reference antibody. A “humaneered” antibody for use in this invention has an immunoglobulin molecule that contains minimal sequence derived from a donor immunoglobulin. Typically, an antibody is “humaneered” by joining a DNA sequence encoding a binding specificity determinant (BSD) from the CDR3 region of the heavy chain of the reference antibody to human V_{H} segment sequence and a light chain CDR3BSD from the reference antibody to a human V_{L} segment sequence. A “BSD” refers to a CDR3-FR4 region, or a portion of this region that mediates binding specificity. A binding specificity determinant therefore can be a CDR3-FR4, a CDR3, a minimal essential binding specificity determinant of a CDR3 (which refers to any region smaller than the CDR3 that confers binding specificity when present in the V region of an antibody), the D segment (with regard to a heavy chain region), or other regions of CDR3-FR4 that confer the binding specificity of a reference antibody. Methods for humaneering are provided in US patent application publication no. 20050255552 and US patent application publication no. 20060134098.

The term “hybrid” when used with reference to portions of a nucleic acid or protein, indicates that the nucleic acid or protein comprises two or more subsequences that are not normally found in the same relationship to each other in nature. For instance, the nucleic acid is typically recombinantly produced, having two or more sequences, e.g., from unrelated genes arranged to make a new functional nucleic acid. Similarly, a hybrid protein refers to two or more subsequences that are not normally found in the same relationship to each other in nature.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The phrase “specifically (or selectively) binds” to an antibody or “specifically (or selectively) immunoreactive with,” when referring to a protein or peptide, refers to a binding reaction where the antibody binds to the protein of interest. In the context of this invention, the antibody typically binds to PcrV with an affinity of 500 nM or less, and has an affinity of 5000 nM or greater, for other antigens.

The terms “identical” or percent “identity,” in the context of two or more polypeptide (or nucleic acid) sequences, refer to two or more sequences or subsequences that are the same or have a specified percentage of amino acid residues (or nucleotides) that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” “Substantially identical” sequences also includes sequences that have deletions and/or additions, as well as those that have substitutions, as well as naturally occurring, e.g., polymorphic or allelic variants, and man-made variants. As described below, the preferred algorithms can account for gaps and the like. Preferably, protein sequence identity exists over a region that is at least about 25 amino acids in length, or more preferably over a region that is 50-100 amino acids in length, or over the length of a protein.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” in some embodiments denotes that a protein gives rise to essentially one band in an electrophoretic gel. Preferably, it means that the protein is at least 85% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables and substitution matrices such as BLOSUM providing functionally similar amino acids are well known in the art. Such conservatively modified variants are in addition to and do not exclude polymorphic variants, interspecies homologs, and alleles of the invention. Typical conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

### INTRODUCTION

The present invention is based on the surprising discovery that antibiotics, e.g., an aminoglycoside such as tobramycin; or a penicillin such as piperacillin, when administered in combination with an antagonist anti-PcrV antibody provides a surprisingly effective therapeutic treatment regimen for P. aeruginosa infections. In some embodiments, the antibiotic is administered at its MTD, as the anti-PcrV antibody treatment dose does not cause further host toxicity. In additional embodiments, the antibiotic is administered at a subefficacious dose and the combined therapeutic effect with the antibody is greater than simple additivity.

In some embodiments, the combination of antibiotic, e.g., an aminoglycoside such as tobramycin, and antibody has the surprising effect of decreasing dissemination of the bacteria into the bloodstream. Thus, in some aspects, the invention provides a method of treating or preventing bacteremia. The ability of an antibiotic/PcrV antibody combination to treat or prevent bacteremia can be determined in animal models, e.g., a mouse model of P. aeruginosa infection as used in the examples provided in the “EXAMPLES” section.

Furthermore, a combination antibiotic/PcrV antibody therapy of the invention increases the sensitivity in vivo of P. aeruginosa to antibiotics to which the strain displays in vitro resistance. Thus, in some embodiments, a combination of PcrV antibody and antibiotic is administered to a subject infected with a P. aeruginosa strain that has some resistance to the antibiotic. Antibiotic sensitivity can be assessed, for example, using assays well known in the art, e.g., a diffusion or broth dilution susceptibility assay. Alternatively, a subject that has a P. aeruginosa infection that has been treated with an antibiotic and hasn't shown clinical improvement can be considered to have in vivo resistance to the antibiotic, or is considered to be infected with a strain that is resistant to the antibiotic in vivo. Accordingly, a dose of antibiotic, which may typically be efficacious in a patient that is infected with a strain of P. aeruginosa that is not resistant to the antibiotic, may be sub-efficacious in a patient that is infected with a strain of P. aeruginosa that has resistance to the antibiotic.

A strain that “has resistance”, or “has some degree of resistance”, to an antibiotic need not be completely resistant to the antibiotic such that antibiotic treatment shows no effect on growth of the bacteria. In the context of this invention, a strain that “has resistance”, or “has some degree of resistance” to an antibiotic typically refers to a strain that exhibits antibiotic resistance in accordance with the Clinical and Laboratory Standards Institute (CLSI) guidelines (see, e.g., publication M02-A10, published Dec. 30, 2008; M07-A8, published Dec. 30, 2008; and M100-S19).

### Methods of Treating a Patient

The invention provides methods of treating a patient that has, or is at risk of having, a P. aeruginosa infection by administering an antagonist PcrV antibody in conjunction with an antibiotic, e.g., an aminoglycoside such as tobramycin; or piperacillin. In some embodiments, the antibody and the antibiotic, e.g., tobramycin or piperacillin, are administered at sub-efficacious doses. In some embodiments, the patient being treated has cystic fibrosis, ventilator-associated pneumonia (VAP), is a neutropenic cancer patient or is a burn patient.

The methods of the invention comprise administering a combination of an antibiotic and a PcrV antibody as a pharmaceutical composition to a P. aeruginosa-infected patient in a therapeutically effective amount using a dosing regimen suitable for treatment of the disease. Administration of antibiotics is well known in the art. The antibody composition can be formulated for use in a variety of drug delivery systems.

The PcrV antibody is provided in a solution suitable for injection into the patient such as a sterile isotonic aqueous solution for injection. One or more physiologically acceptable excipients or carriers can also be included in the compositions for proper formulation. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990). The antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

The antibiotic and anti-PcrV antibody are administered to a patient having a P. aeruginosa infection in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose include amelioration of symptoms of infection in the patient, or a decrease in the levels of P. aeruginosa in the patient. Amounts effective for this use will depend upon the severity of the disease and the general state of the patient's health, including other factors such as age, weight, gender, administration route, etc. Single or multiple administrations of the antibody and antibiotic may be administered depending on the dosage and frequency as required and tolerated by the patient. In any event, the methods provide a sufficient quantity of PcrV antibody and antibiotic to effectively treat the patient.

In some embodiments, the antibody is administered with the antibiotic to a patient who has been treated with the antibiotic, but where the antibiotic has not been clinically effective. In the context of this invention, clinical effectiveness refers to the ability to reduce the number of bacteria in a sample, e.g., blood or sputum, from an infected patient. Thus, an antibiotic is not clinically effective if a sample from a patient exhibit about the same, or an increase in, the number of bacteria present in the sample.

In some embodiments, the antibody/antibiotic combination is administered to a patient in whom the maximum tolerated dose of the antibiotic has not been clinically effective. The maximum tolerated dose is determined clinically and is the highest dose that does not cause unacceptable side effects.

In some embodiments, the antibiotic and anti-PcrV antibody combination does not have increased toxicity when administered to a subject compared to when the same antibiotic is administered alone. Thus, for example, a patient may be treated with an antibiotic and experience an adverse side effect from the antibiotic, but the combination does not increase the toxicity of the antibiotic as it relates to the adverse side effect. In some embodiments, the antibody and antibiotic may have complementary toxicities, where toxicity of one agent, such as the antibody, is not exacerbated by side effects of the other agent, e.g., the anti-PcrV antibody. Various toxic effects of antibiotics are known.

In some embodiments, the antibody/antibiotic combination is administered to a patient at risk for a P. aeruginosa infection. Such patients include, e.g., a patient in a hospital setting such as an intensive care unit where another patient has a P. aeurginosa infection; a patient who has been on a ventilator for four days or longer; or a patient with a disease such as cystic fibrosis where the patient has an increased chance of being infected with P. aeruginosa, e.g., the patient is exposed to an individual infected with P. aeruginosa.

The antibody and antibiotic may also be administered in combination with other therapies to treat the P. aeruginosa infection. In the combination treatment of the invention, the antibody can be administered before or after the antibiotic, e.g., within the same day, or within the same week, or at the same time. In some embodiments, the antibody is administered concurrently with the antibiotic after one or more initial treatments with the antibiotic alone.

The antibody can be administered by injection or infusion through any suitable route including but not limited to intravenous, subcutaneous, intramuscular, intratracheal, or intraperitoneal routes. In some embodiments, the antibody may be administered by insufflation. In an exemplary embodiment, the antibody may be stored at 10 mg/ml in sterile isotonic aqueous saline solution for injection at 4° C. and is diluted in either 100 ml or 200 ml 0.9% sodium chloride for injection prior to administration to the patient. The antibody is administered by intravenous infusion over the course of 1 hour at a dose of between 0.2 and 10 mg/kg. In other embodiments, the antibody is administered by intravenous infusion over a period of between 15 minutes and 2 hours. In still other embodiments, the administration procedure is via sub-cutaneous bolus injection.

The dose of antibody is chosen in order to provide effective therapy for the patient and is in the range of less than 0.1 mg/kg body weight to 25 mg/kg body weight or in the range 1 mg-2 g per patient. Preferably the dose is in the range 1-10 mg/kg or approximately 50 mg-1000 mg/patient. The dose may be repeated at an appropriate frequency which may be in the range once per day to once every three months, depending on the pharmacokinetics of the antibody (e.g. half-life of the antibody in the circulation) and the pharmacodynamic response (e.g. the duration of the therapeutic effect of the antibody). In some embodiments, the in vivo half-life of between about 7 and about 25 days and antibody dosing is repeated between once per week and once every 3 months. In other embodiments, the antibody is administered approximately once per month.

In further embodiments, the antibody is PEGylated. For example, an antibody of the invention may be PEGylated, e.g., using methods as described herein, and administered to a patient infected with P. aeruginosa. By way of further example, the PEGylated antibody may be an antibody fragment, such as a Fab′ fragment.

Methods of administering antibiotics are well known in the art. For example, the antibiotic is typically administered orally or by injection, for example, intravenously, subcutaneously, intramuscularly, parenterally, intratracheally or using spinal or epidermal routes. In some embodiments, e.g., in certain embodiments where an aminoglycoside such as tobramycin is administered, the antibiotic can be aerosolized for administration by inhalation.

### Antibiotics

In some embodiments, the antibiotic that is administered in combination with an anti-PcrV antibody is an aminoglycoside antibiotic such as tobramycin. Aminoglycoside antibiotics refers to both synthetic and natural antibiotics isolated from species of Streptomyces and Micromonospora. These antibiotics include gentamicin, netilmicin, tobramycin, kanamycin, neomycin, amikacin, arbekacin, azithromycin, streptomycin, netilmicin, paromomycin, rhodostreptomycin, and apramycin. One of the major disadvantages of aminoglycosides is that they can induce fairly severe side effects. The present invention provides a treatment method employing such an antibiotic where the antibiotic can be administered at lower doses than when the antibiotic alone is administered.

In some embodiments, the methods of treating P. aeruginosa infection comprises administering an anti-PcrV antibody in conjunction with an antibiotic such as a cephalosporin, e.g., ceftazidime, cefepime, cefpirome, cefuroxime, ceftriaxone, cefotaxime; or a quinalone, e.g., a fluoroquinalone, such as ciprofloxacin, or levofloxacin; or a ureidopenicillin, e.g., penicillin, pipericillin or ticarcillin, azlocillin; carbapenems, e.g., meropenem, imipenem; polymyxins, e.g., polymyxin B and colistin), and monobactams, e.g., aztreonam. As understood in the art a ureidopenicillin antibiotic such as piperacillin, is typically administered in a format that includes a penicillinase inhibitor such as tazobactam. Other antibiotics that can be used include sulfonamides, tetracyclines, glycylcyclines, e.g., tigecycline, and macrolides. In some embodiments, an antibiotic that induces the TTSS, e.g., a tetracycline (Linares et al., Proc. Natl. Acad. Sci. USA 103:19484-19489; 2006), is used.

### Anti-PcrV Antibodies

The invention relates to methods of treatment of P. aeruginosa infection using antibiotics in combination with antibodies that bind with high affinity to the PcrV antigen from P. aeruginosa and are typically functional antagonists of the Type III secretion system. This section provides examples of antibodies, e.g., humaneered antibodies that can be employed in the therapeutic regimens of the invention.

Antibodies for use in the invention typically comprise variable regions with a high degree of homology to human germ-line V_{H} and V_{L} sequences. The CDR3 sequences of the heavy and light chains comprise a pair of binding specificity determinants (BSD) from the monoclonal anti-PcrV antibody Mab166 (Frank et al., J. Infectious Dis. 186: 64-73, 2002; and U.S. Pat. No. 6,827,935) and the antibodies of the invention compete with Mab166 for binding to a neutralizing epitope on the PcrV protein (see, e.g., U.S. Pat. No. 6,827,935).

In some embodiments, antibodies for use in the invention have a minimal essential binding specificity determinant in CDRH3 that has the amino acid sequence NRGDIYYDFTY. In some embodiments, such an antibody has a heavy chain CDR3 sequence NRGDIYYDFTYA(M/F)DX, where X is I, S, or Q.

In some embodiments, antibodies for use in the invention have a minimal essential binding specificity determinant in CDRL3 that has the amino acid FWXTP (where X may be either S or G). Complete V-regions are generated in which the BSD forms part of the CDR3 and additional sequences are used to complete the CDR3 and add a FR4 sequence. Typically, the portion of the CDR3 excluding the BSD and the complete FR4 are comprised of human germ-line sequences. In preferred embodiments, the CDR3-FR4 sequence excluding the BSD differs from human germ-line sequences by not more than 2 amino acids on each chain.

The human germline V-segment repertoire consists of 51 heavy chain V-segments, 401c light chain V-segments, and 31λ light chain V-segments, making a total of 3,621 germline V-region pairs. In addition, there are stable allelic variants for most of these V-segments, but the contribution of these variants to the structural diversity of the germline repertoire is limited. The sequences of all human germ-line V-segment genes are known and can be accessed in the V-base database (on the worldwide web at vbase.mrc-cpe.cam.ac.uk), provided by the MRC Centre for Protein Engineering, Cambridge, United Kingdom (see, also Chothia et al., 1992, J Mol Biol 227:776-798; Tomlinson et al., 1995, EMBO J. 14:4628-4638; Cook et al. (1995) Immunol. Today 16: 237-242 and Williams et al., 1996, J Mol Biol 264:220-232); or the international ImMunoGeneTics database (IMGT). These sequences can be used as reference sources for the human germline segments of the antibodies of the invention.

Antibodies or antibodies fragments as described herein can be expressed in prokaryotic or eukaryotic microbial systems or in the cells of higher eukaryotes such as mammalian cells.

An antibody that is employed in the invention can be in any format. For example, in some embodiments, the antibody can be a complete antibody including a constant region, e.g., a human constant region, or can be a fragment or derivative of a complete antibody, e.g., a Fab, Fab′, F(ab′)₂, scFv, Fv, or a single domain antibody, such as a nanobody or a camelid antibody.

### II. Heavy Chains

A heavy chain of an anti-PcrV antibody for use in combination with an antibiotic comprises a heavy-chain V-region that comprises the following elements:

1) human heavy-chain V-segment sequences comprising FR1-CDR1-FR2-CDR2-FR3

2) a CDRH3 region comprising the amino acid sequence NRGDIYYDFTY

3) a FR4 contributed by a human germ-line J-gene segment.

Examples of V-segment sequences that support binding to PcrV in combination with a CDR3-FR4 segment described above together with a complementary V_{L} region are shown in FIG. 8. The V-segments can be from the human VH1 or VH3 sub-classes. In some embodiments, the V-segment is a human V_{H}3 sub-class segment that has a high degree of amino-acid sequence identity with the germ-line segment VH3-30.3. For example the V-segment differs by not more than fifteen residues from VH3-30.3 and preferably not more than seven residues.

The FR4 sequence of the antibodies of the invention is provided by a human J segment. There are six heavy chain JH-regions numbered 1 through 6. Thus, the FR4 sequences can be provided by a JH1, JH2, JH3, JH4, JH5 or JH6 gene segment. Typically, the FR4 region of an antibody of the invention has at least 90%, often at least 91%, 92%, 93%, 94%, 95% 96%, 97%, 98%, 99%, or 100% identity, to the FR4 region of the human germline J segment that provides the FR4.

In some embodiments, the FR4 sequence is provided by a human germ-line JH3 segment and has a sequence WGQGTMVTVSS. In other embodiments, the FR4 is provided by a human germ-line JH6 segment and has the sequence WGQGTTVTVSS.

The CDRH3 also comprises sequences that are derived from a human J-segment. Typically, the CDRH3-FR4 sequence excluding the BSD differs by not more than 2 amino acids from a human germ-line J-segment. In typical embodiments, the J-segment sequences in CDRH3 are from the same J-segment used for the FR4 sequences. Thus, in some embodiments, the CDRH3-FR4 region comprises the BSD and a complete human JH3 germ-line gene segment. Exemplary combinations of CDRH3 and FR4 sequences are shown below, in which the BSD is in bold and human germ-line J-segment residues are underlined:

| CDR3 | |
|---|---|
| NRGDIYYDFTYAFDIWGQGTMVTVSS | (FR4 = JH3) |
| NRGDIYYDFTYAMDIWGQGTMVTVSS | (FR4 = JH3) |
| NRGDIYYDFTYAMDIWGQGTTVTVSS | (FR4 = JH6) |

In some embodiments, an antibody of the invention comprises a V-segment that has at least 90% identity, or at least 91%, 92% 93%, 94%, 95%, 965, 97%, 98%, 99%, or 100% identity to the germ-line segment VH3 30.3 or to a germlineVH1-18 segment; or to one of the V-segments of the V_{H} regions shown in FIG. 8, such as a V-segment portion of SEQ ID NOs 1, 3, 5, 7, 9, 11, 13, 15, 17, 19, 21, 23, 25, 26, 27, 29, and 35.

In some embodiments, the V-segment of the VH region has a CDR1 and/or CDR2 as shown in FIG. 8. For example, an antibody of the invention may have a CDR1 that has the sequence TAGMH, SYGIH, SYGMH, SYPLH, or NYPMH; or a CDR2 that has the sequence VIWYNGKEISYADSVKG, FISYDGSEKYYASSVKG, or VISYDGSEKWYADSVKG. In some embodiments, the CDR2 of the VH region has a negatively charged amino acid positioned in about the middle, e.g., at position 8 or 9 of the CDR2.

In particular embodiments, an antibody has both a CDR1 and a CDR2 from one of the V_{H} region V-segments shown in FIG. 8 and a CDR3 that comprises NRGDIYYDFTY, e.g., NRGDIYYDFTYAFDI or NRGDIYYDFTYAMDI. Thus, an anti-PcrV antibody of the invention, may for example, have a CDR3-FR4 that has the sequence NRGDIYYDFTYAFDIWGQGTMVTVSS, NRGDIYYDFTYAMDIWGQGTMVTVSS, or NRGDIYYDFTYAMDIWGQGTTVTVSS. In other embodiments, the antibody may comprise a CDR3 that has the sequence NRGDIYYDFTYA(M/F)D(Q/S).

### III. Light Chains

A light chain of an anti-PcrV antibody for use in the invention comprises at light-chain V-region that comprises the following elements:

1) human light-chain V-segment sequences comprising FR1-CDR1-FR2-CDR2-FR3

2) a CDRL3 region comprising the sequence FWXTP (where X may be S or G)

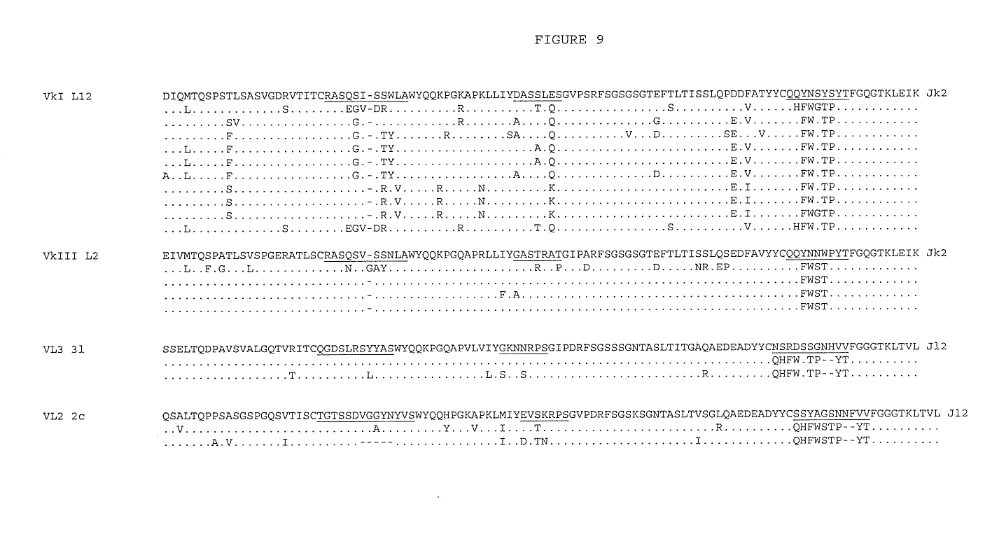

3) a FR4 contributed by a human germ-line J-gene segment. The V_{L} region comprises either a Vlambda or a Vkappa V-segment. Examples of Vlambda and Vkappa sequences that support binding in combination with a complementary V_{H}-region are provided in FIG. 9. Vkappa segments are cloned upstream of the human germ-line JK2 segment and Vlambda segments are cloned upstream of the germ-line JL2 segment.

The CDRL3 sequence comprises a V-segment and J-segment derived sequences. In typical embodiments, the J-segment sequences in CDRL3 are from the same J-segment used for FR4. Thus, may differ by not more than 2 amino acids from human kappa germ-line V-segment and J-segment sequences. In some embodiments, the CDRL3-FR4 region comprises the BSD and the complete human JK2 germ-line gene segment. Exemplary CDRL3-FR4 combinations for kappa chains are shown below in which the BSD is shown in bold and JK2 sequences are underlined:

CDR3
QQFWSTPYTFGQGTKLEIK (JK2)
QHFWGTPYTFGQGTKLEIK (JK2)

A preferred CDR3-FR4 for lambda chains is shown below in which the BSD is shown in bold and the JL2 sequences are underlined:

CDR3
QHFWSTPYTFGGGTKLTVL (JL2)

The FR4 sequence of the antibodies of the invention is provided by a human J segment. There are five human JKappa-region segments labeled 1 though 5 and four JLambda-region segments labeled 1, 2, 3 and 7. Thus, the FR4 sequences can be provided by any of these germline sequences. Typically, the FR4 region of an antibody of the invention has at least 90%, often at least 91%, 92%, 93%, 94%, 95% 96%, 97%, 98%, 99%, or 100% identity, to the FR4 region of the human germline J segment that provides the FR4.

The Vkappa segments are typically of the VKI or VKIII sub-class. In some embodiments, the segments have at least 80% sequence identity to a human germline VKI or VKIII subclass, e.g., at least 80% identity to the human germ-line VKI L12 sequence or to human germline VKIII L2 or VKIIIA11 sequence. For example, the Vkappa segment may differ by not more than 18 residues from VKI L12, or 12 residues from VKIII A11 or VKIII L2. In other embodiments, the V_{L} region V-segment of an antibody of the invention has at least 85% identity, or at least 90%, 91%, 92%, 93%, 94%, 95%, 96%, 97%, 98%, 99%, or 100% identity to the human germline VKI L12, or to the human germline VkIII L2 sequence, or to human germline VKIII A11 sequence, or to a kappa V-segment sequence of a V_{L} region shown in FIG. 9, for example, the V-segment sequence of SEQ ID NOs. 2, 4, 6, 8, 10, 12, 14, 16, 18, 20, 22, 24, or 37.

In some embodiments, the V-segment of the V_{L} corresponds to a human germline Vlambda segment. Thus, in some embodiments, the V-segment has at least 85% identity, or at least 90%, 91%, 92%, 93%, 94%, 95%, 96%, 97%, 98%, 99%, or 100% identity to a Vlambda V-segment of a V_{L} region of FIG. 9, such as the V-segment sequence of SEQ ID NOs. 28, 30, 32, or 34.

In some embodiments, the V-segment of the V_{L} region has a CDR1 and/or CDR2 as shown in FIG. 9. For example, an antibody of the invention may have a CDR1 sequence of RASQGISTYLA, RASQGISSWLA, RASQSISRWVA, RASQGISTYLA, or RASEGVDRWLA or CDR2 sequence AASSLQS, DASSLKS, AASSLQS, DASALQS, or DASTLQS. In other embodiments, the antibody may have a CDR1 sequence of QGDSLRSYYA, TGTSSDVGAYNYVS, or TGTSSDYV; or a CDR2 sequence GKNNRPS, EVTKRPS, or DVTNRPS.

In particular embodiments, an anti-PcrV antibody of the invention may have a CDR1 and a CDR2 in a combination as shown in one of the V-segments of the V_{L} regions set forth in FIG. 9 and a CDR3 sequence that comprises FWXTP, where X is S or G, e.g., the CDR3 may be QQFWSTPYT, QHFWGTPYT, or QHFWSTPYT. In some embodiments, such an anti-PcrV antibody may comprise an FR4 region that is FGQGTKLEIK or FGGGTKLTVL. Thus, an anti-PcrV antibody of the invention, can comprise, e.g., both the CDR1 and CDR2 from one of the V_{L} regions shown in FIG. 9 and a CDR3-FR4 region that is QFWSTPYTFGQGTKLEIK, QHFWGTPYTFGQGTKLEIK, or QHFWSTPYTFGGGTKLTVL.

### IV. Preparation of PcrV Antibodies

An anti-PcrV antibody of the invention may comprise any of the V_{H} regions of SEQ ID NOs. 1, 3, 5, 7, 9, 11, 13, 15, 17, 19, 21, 23, 25, 26, 27, 29, or 35 in combination with any of the V_{L} regions of SEQ ID NOs. 2, 4, 6, 8, 10, 12, 14, 16, 18, 20, 22, 24, 28, 30, 32, 34, 36 or 37.

An antibody may be tested to confirm that the antibody retains the activity of antagonizing the Type III secretion system. The antagonist activity can be determined using any number of endpoints, including cytotoxicity assays. Exemplary assays are described, e.g., in U.S. Pat. No. 6,827,935. An antibody that is administered to treat P. aeruginosa infection preferably retains at least 75%, preferably 80%, 90%, 95%, or 100%, of the Type III secretion pathway antagonist activity of Mab166 (U.S. Pat. No. 6,827,935).

A high-affinity antibody may be identified using well known assays to determine binding activity and affinity. Such techniques include ELISA assays as well as binding determinations that employ surface plasmon resonance or interferometry. For example, affinities can be determined by biolayer interferometry using a ForteBio (Mountain View, Calif.) Octet biosensor.

Antibodies of the invention typically compete with Mab166 for binding to PcrV. The region of PcrV to which Mab166 binds has been identified (U.S. Pat. No. 6,827,935). PcrV or a fragment thereof that binds Mab166 can be employed in a competitive binding assay. The ability of an antibody described herein to block or compete with Mab166 for binding to PcrV indicates that the antibody binds to the same epitope as Mab166 or to an epitope that is close to, e.g., overlapping, with the epitope that is bound by Mab166. In other embodiments an antibody described herein, e.g., an antibody comprising a V_{H} and V_{L} region combination as shown in Table 1, can be used as a reference antibody for assessing whether another antibody competes for binding to PcrV. A test antibody is considered to competitively inhibit binding of a reference antibody, if binding of the reference antibody to the antigen is reduced by at least 30%, usually at least about 40%, 50%, 60% or 75%, and often by at least about 90%, in the presence of the test antibody. Many assays can be employed to assess binding, including ELISA, as well as other assays, such as immunoblots.

In some embodiments, the anti-PcrV antibody need not antagonize the Type III secretion sequences. For example, antibodies of the invention that bind to PcrV can recruit multiple cell types of the immune system to stimulate phagocytosis by macrophages, antibody directed cellular cytotoxicity (ADCC) by macrophages or NK cells, activation of the complement cascade, and/or generation of the oxidative burst by neutrophils, thereby causing bacterial, i.e., P. aeruginosa, death. Furthermore, all antibody variable regions are capable of catalyzing redox reactions from singlet oxygen provided by activated neutrophils, leading to the generation of a variety of highly potent oxidizing agents directly harmful to bacteria (see, e.g., Wentworth et al., Proc. Natl. Acad. Sci. USA 97:10930-10935, 2000), including ozone, a potent antibacterial agent which also stimulates inflammatory responses (see, e.g., Babior et al., Proc. Natl. Acad. Sci. USA 100:3031-3034, 2003). Indeed, inflammation induced by complement activation and ozone generation has the potential to recruit additional elements of the immune system to further boost immunity. Such antibodies typically have an affinity of 50 nM or less, typically less than about 10 nM.

Non-neutralizing and neutralizing anti-PcrV antibodies used in combination with antibiotics provide a strong therapeutic effect.

Methods for the isolation of antibodies with V-region sequences close to human germ-line sequences have previously been described (US patent applications 20050255552 and 20060134098). Antibody libraries may be expressed in a suitable host cell including mammalian cells, yeast cells or prokaryotic cells. For expression in some cell systems, a signal peptide can be introduced at the N-terminus to direct secretion to the extracellular medium. Antibodies may be secreted from bacterial cells such as E. coli with or without a signal peptide. Methods for signal-less secretion of antibody fragments from E. coli are described in US patent application 20070020685.

To generate a PcrV-binding antibody, one of the V_{H}-regions of the invention is combined with one of the V_{L}-regions of the invention and expressed in any of a number of formats in a suitable expression system. Thus the antibody may be expressed as a scFv, Fab, Fab′ (containing an immunoglobulin hinge sequence), F(ab′)₂, (formed by di-sulfide bond formation between the hinge sequences of two Fab′ molecules), whole immunoglobulin or truncated immunoglobulin or as a fusion protein in a prokaryotic or eukaryotic host cell, either inside the host cell or by secretion. A methionine residue may optionally be present at the N-terminus, for example, in polypeptides produced in signal-less expression systems. Each of the V_{H}-regions described herein may be paired with each of the V_{L} regions to generate an anti-PcrV antibody. For example, VH3 1080-2F was identified from the library paired with two different lambda light chains (1080-2F and 1080-11E). The kappa chain 1069-3F was identified paired with VH3 1069-3F and with VH3 1100-3. Exemplary combinations of heavy and light chains are shown in the Table 1.

TABLE 1
Exemplary antibody heavy-chain
and light-chain combinations

| VH | |
|---|---|
| | Vkappa |
| SEQ ID NO: 1 | SEQ ID NO: 2 |
| SEQ ID NO: 11 | SEQ ID NO: 12 |
| SEQ ID NO: 3 | SEQ ID NO: 12 |
| SEQ ID NO: 7 | SEQ ID NO: 8 |
| SEQ ID NO: 9 | SEQ ID NO: 10 |
| SEQ ID NO: 5 | SEQ ID NO: 6 |
| SEQ ID NO: 13 | SEQ ID NO: 37 |
| SEQ ID NO: 21 | SEQ ID NO: 18 |
| SEQ ID NO: 17 | SEQ ID NO: 18 |
| SEQ ID NO: 26 | SEQ ID NO: 24 |
| SEQ ID NO: 25 | SEQ ID NO: 24 |
| SEQ ID NO: 23 | SEQ ID NO: 24 |
| SEQ ID NO: 29 | SEQ ID NO: 20 |
| SEQ ID NO: 35 | SEQ ID NO: 36 |
| | Vlambda |
| SEQ ID NO: 29 | SEQ ID NO: 28 |
| SEQ ID NO: 29 | SEQ ID NO: 30 |
| SEQ ID NO: 29 | SEQ ID NO: 34 |
| SEQ ID NO: 3 | SEQ ID NO: 32 |

In many embodiments, the antibodies of the invention antagonize the P. aeruginosa type III secretion system and typically exhibit high affinity binding to PcrV. High affinity binding between an antibody and an antigen exists if the affinity of the antibody is less than 500 or 100 nM, for example, less than 50 nM or less than 25 nM, or less than 10 nM, or less than 1 nM, e.g., less than about 100 pM. The antibodies of the invention typically have an affinity of 50 nM or less, often 10 nM or less, when assayed as Fabs, e.g., using ELISA, surface plasmon resonance assays, or interferometry. Table 1 provides examples of such antibodies.

In some embodiments, an antibody of the invention is more potent in a cellular cyototoxicity assay than Mab166.

Antibodies may be produced using any number of expression systems, including both prokaryotic and eukaryotic expression systems. Many such systems are widely available from commercial suppliers. In embodiments in which an antibody comprises both a V_{H} and V_{L} region, the V_{H} and V_{L} regions may be expressed using a single vector, e.g., in a dicistronic expression unit, or under the control of different promoters. In other embodiments, the V_{H} and V_{L} region may be expressed using separate vectors. The antibodies of the invention may be expressed with or without a methionine at the N-terminus. Thus, a V_{H} or V_{L} region as described herein may optionally comprise a methionine at the N-terminus.

An antibody of the invention may be produced in any number of formats, including as a Fab, a Fab′, a F(ab′)₂, a scFv, or a dAB. An antibody of the invention can also include a human constant region. The constant region of the light chain may be a human kappa or lambda constant region. The heavy chain constant region is often a gamma chain constant region, for example, a gamma-1, gamma-2, gamma-3, or gamma-4 constant region. In other embodiments, the antibody may be an IgA.

In some embodiments, the antibody is “non-immunogenic” when administered to a human. The term “non-immunogenic” as used here refers to a PcrV antibody of the invention that does not provoke antibody production against the anti-PcrV antibody when administered to a human. Antibodies can be assessed for immunogenicity using known assays, e.g., an electrochemiluminescence immunoassay described in example 5. Such assays detect the level of antibodies present in a patient, e.g., in a serum sample from the patient, that react with the anti-PcrV antibody that is administered to the patient. An assay is considered to show that the antibody is non-immunogenic when no detectable antibody to the anti-PcrV antibody is present in the sample, e.g., in comparison to a control sample from an individual that was not administered the antibody.

### V. PEGylation of Antibodies

In some embodiments, e.g., where the antibody is a fragment, the antibody can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007).

In some embodiments, the antibodies of the invention are in the form of a Fab′ fragment. A full-length light chain is generated by fusion of a V_{L}-region to human kappa or lambda constant region. Either constant region may be used for any light chain; however, in typical embodiments, a kappa constant region is used in combination with a Vkappa variable region and a lambda constant region is used with a Vlambda variable region.

The heavy chain of the Fab′ is a Fd fragment generated by fusion of a V_{H}-region of the invention to human heavy chain constant region sequences, the first constant (CH1) domain and hinge region. The heavy chain constant region sequences can be from any of the immunoglobulin classes, but is often from an IgG, and may be from an IgG1, IgG2, IgG3 or IgG4. The Fab′ antibodies of the invention may also be hybrid sequences, e.g., a hinge sequence may be from one immunoglobulin sub-class and the CH1 domain may be from a different sub-class. In a preferred embodiment, the heavy chain constant region including the CH1 domain and hinge sequence is from human IgG1.

The Fab′ molecule can be PEGylated using known methods. The hinge region of the heavy chain contains cysteine residues suitable for conjugation to a polyethylene glycol derivative. The hinge sequence may be the complete natural hinge region of an immunoglobulin heavy chain or may be truncated by one or more amino-acids. In some embodiments, the hinge region may be a modified or synthetic sequence. In further embodiments, the hinge is a natural immunoglobulin hinge sequence and contains two cysteine residues.

In some embodiments, Fab′ molecules can be conjugated by site-specific conjugation to maleimide derivatives of methoxy polyethylene glycol (mPEG-mal). The mPEG-mal can have, for example, an average molecular mass of between 10 and 40 kD. The PEG may be branched PEG or linear PEG. In some embodiments, the mPEG-mal is a linear molecule and has an approximate molecular weight of 30 kD. One or more molecules of mPEG-mal is conjugated to each Fab′ molecule. The mPEG molecules are conjugated via thioether linkages between the maleimide moiety of mPEG-mal and one or more of the cysteine residues in the hinge region of the Fab′ heavy chain to form the PEGylated Fab′ molecule. The mPEG-mal is conjugated in suitable buffer and under conditions suitable for thioether formation using methods known in the art for conjugation of maleimide derivatives to thiol-groups on proteins.

The Fab′ may be produced from the expression system in a form in which the hinge cysteine groups are in an oxidized form. In this case, the Fab′ may be subjected to a reduction step prior to conjugation. Reducing agents suitable for generation of free hinge thiols and methods for selective reduction of hinge cysteines are known in the art and include the use of dithiothreitol (DTT), beta-mercapto-ethanol, beta-mercapto-ethylamine (MEA) and non-thiol reducing agents such as tris(2-carboxyethyl)phosphine. In some embodiments, the reduction is carried out under conditions such that the hinge cysteines are selectively reduced and PEGylation occurs predominantly at the hinge. Typically, the PEGylated Fab′ comprises two molecules of mPEG due to PEGylation of both cysteine residues in the hinge. In some embodiments, a mutation may be introduced into the hinge region to replace one of the cysteine residues with another amino acid. Derivatization of such a mutant with mPEG-mal leads to the generation of mono-PEGylated Fab′.

Other methods of PEGylation, for example, where the PEG is not introduced at a hinge are also known. For example, Humphreys et al., supra, describe methods for PEGylation of cysteine residues outside the hinge region by disruption of the interchain disulphide bond between the heavy and light chain of a Fab.

Methods for purification of PEGylated Fab′ are known in the art. Such methods include, for example, size-exclusion or ion-exchange chromatography.

### EXAMPLES

### Example 1

### Effects of Antibiotic, Anti-PcrV Antibody Combination Therapy In Vivo

### Material and Methods:

Antibiotics and Mab166 preparations. Four anti-Pseudomonal antibiotics were used in this example: ciprofloxacin (Bayer HealthCare, N.J.), ceftazidime (GSK, UK), tobramycin (Abraxis, Ill.) and piperacillin (Wyeth, Pa.). Antibiotic solutions were made immediately prior to use. Anti-PcrV antibody Mab166 was diluted to a working concentration of 3 mg ml⁻¹ in sterile PBS.

Bacterial administration and lung injury measurement in mice. Following anesthesia with avertin (250 mg kg⁻¹; ip), mice were inoculated with a volume of fifty μl of bacterial working stock (1.5×10⁶ CFU), which was instilled into the left lung through the trachea using a 27G gavages needle. Mice were allowed to recover for 15 minutes prior to being returned to their cages. Mice were active and appeared normal 30 min post inoculation. Rectal temperature was recorded hourly for the initial 12 hours post bacterial instillation followed by daily measurements for 7 days. Survival time was recorded for each mouse.

To examine lung injury (in the tobramycin treatment groups), three mice in each group were euthanized 8 and 24 hours post bacterial instillation. Blood samples were collected aseptically in sodium-citrate tubes using right ventricle punctures following thoracotomies. Lungs were removed, weighed and homogenized for lung injury measurements. Excess lung water (ELW) and wet/dry weight ratio were calculated as previously described (Hijazi, et al. Sem. in Resp. and Crit. Care Med. 21:245-262, 2000).

Antibiotics and PcrV antibody administration. Mab166 (300 μg per mouse in a total volume of 100 μl) was administrated intravenously through tail vein 1 hour prior to P. aeruginosa instillation. This sub-optimal concentration of Mab166 was chosen since previous studies using 400 μg per mouse had demonstrated full protection (100% survival). All antibiotics were administrated intraperitoneally 1 hour after P. aeruginosa instillation. Four doses of each antibiotic were tested in mice to determine the dose needed to produce sub-optimal survival rates (40-60%) in mice infected with 1.5×10⁶ PA103 CFU, which is a 3× lethal amount of bacteria. The dose and timing of antibiotic administration was as follows: ciprofloxacin (100 mg kg⁻¹; Q8H), tobramycin (3.3 mg kg⁻¹; Q8H) and ceftazidime (1000 mg kg⁻¹; Q8H) for the duration of this study.

Bacterial enumeration. Lungs were removed aseptically from the thoracic cavity, placed in 1 ml of sterile PBS and homogenized. Spleens were aseptically removed and homogenized in 1 ml of PBS and blood was drawn from right ventricle puncture. All samples were serially diluted in sterile PBS and plated on P. aeruginosa isolation agar prior to overnight incubation at 37° C. Plates with cell counts between 30 and 300 were enumerated and mean CFU ml⁻¹ of triplicate counts was calculated for each sample.

Myeloperoxidase (MPO) assay. Five hundred μl of the blood collected for bacterial enumeration was centrifuged at 10,000 rpm for 10 min at 4° C. to obtain plasma. The Murine Myeloperoxidase ELISA kit (Cell Science, Mass.) was used to measure MPO activity in mouse plasma samples according to the manufacturer's instructions.

Mab166 titer. Plasma obtained as described above was used to determine Mab166 titer using an antigen-binding ELISA.

PA103 antibiotic sensitivity Dade Behring MicroScan Neg Combo plates were used to determine the antibiotic sensitivity of PA103 by conventional antibiotic sensitivity testing according to the manufacturers instructions.

Statistics. Kaplan-Meier plots were generated for survival analysis and ANOVA was used to compare lung injury score, bacterial CFU's and MPO activity in various treatment groups at specific time points; P 0.05 was considered significant.

### Results.

### Mab166/Antibiotic Combination Therapy Improves Mouse Survival.

400 μg of Mab166 administered into the tail vein resulted in 100% survival of mice infected with 1.5×10⁶ CFU of P. aeruginosa PA103 compared to PBS injected controls (data not shown). For this study, sub-optimal protection was necessary to determine if administration of Mab166 in combination with antibiotic administration improved mouse survival. Therefore, a dose-dependent survival curve was performed using PBS alone and 3 concentrations of Mab166 (100, 200 and 300 μg) injected through the tail vein of mice 1 hour prior to P. aeruginosa instillation. Mouse survival increased with increasing concentrations of Mab166 (Table 2), with 300 μg Mab166 providing a mean survival time of approximately 36 hours (±5.5 h). As this concentration would permit clear differences in survival to be determined between the treatment and control groups, it was chosen for all subsequent studies.

TABLE 2
Mab166 dose-dependent acute infection survival time.

| Mab166 concentration (μg) | Mean survival time (hours ± SEM^{a}) |
|---|---|
| 0 | 14 ± 2.3 |
| 100 | 24.6 ± 2.0 |
| 200 | 32 ± 1.6 |
| 300 | 35.6 ± 5.5 |

^{a}SEM, Standard error of the mean

A longitudinal experimental design was set up to determine if administration of Mab166 in combination with anti-Pseudomonal antibiotics improved mouse survival in an acute model of murine airway infection. For each antibiotic examined, animals were separated into four groups, a control (no treatment) and three treatment groups (Mab166 alone; antibiotic alone; Mab166 and antibiotic in combination). Four antibiotics, ceftazidime, ciprofloxacin, tobramycin, and piperacillin/tazobactam combination were tested using this experimental design. The acutely infectious strain P. aeruginosa PA103 was used for this study. Conventional antibiotic resistance testing of this strain and interpretation of the results using Clinical and Laboratory Standards Institute (CLSI) guidelines demonstrated that it was susceptible to ceftazidime, ciprofloxacin, and tobramycin antibiotics used in this study. The strain had some resistance to piperacillin.

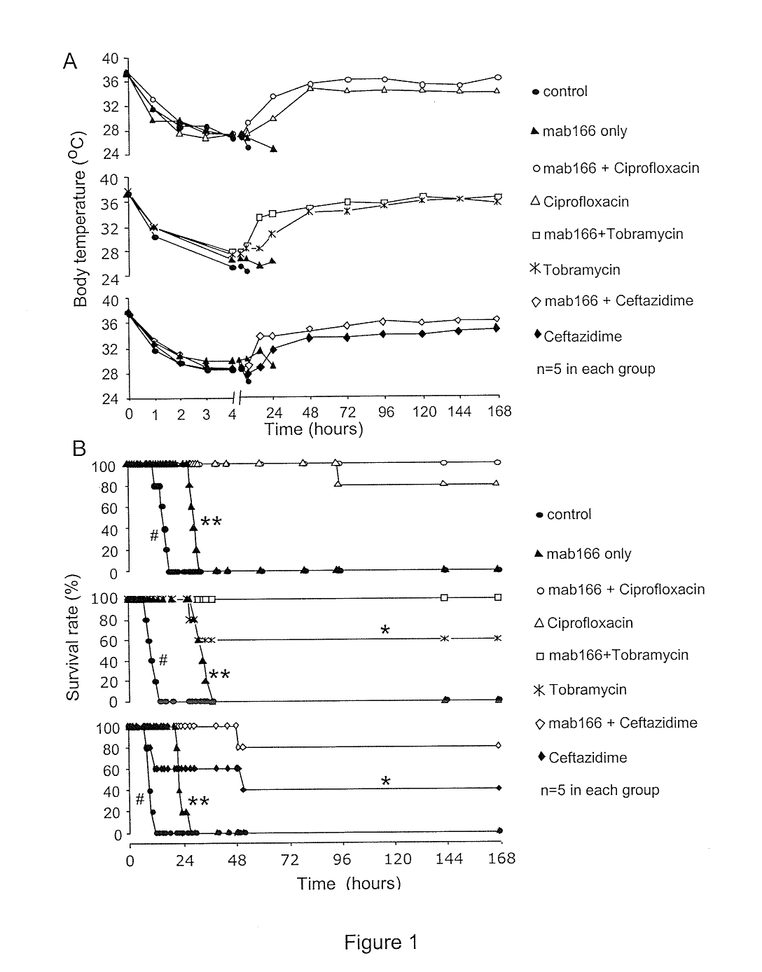

Mice in all groups demonstrated a rapid (within 4 hours) decrease in temperature following instillation of P. aeruginosa. While the temperature of mice in the control group dropped continuously until death, animals in the Mab166 alone treated group exhibited a sustained, albeit low temperature in the hours prior to death (FIG. 1A). Body temperature of the mice in the antibiotic alone or Mab166/antibiotic combination treatment groups recovered to near normal temperatures after the initial decrease. Interestingly, mice in the Mab166/antibiotic treatment group consistently exhibited marginally higher body temperatures that were closer to normal compared to those exhibited by the antibiotic alone treatment group (FIG. 1A).

Survival curves demonstrated that the control mice in each experiment consistently died approximately 14 hours post-infection (FIG. 1B; Table 2). Mab166 administration alone substantially prolonged mouse survival time compared to animals in the untreated control groups (FIG. 1B; Table 2). Mice in the antibiotic treated group demonstrated further enhanced survival compared to both the control and Mab166 treated groups (FIG. 1B). Improved survival was antibiotic dependent; mice treated with ciprofloxacin, tobramycin and ceftazidime demonstrated an 80%, 60% and 40% survival rate respectively over a 7 day period. This differential may be due to pharmacokinetic and pharmacodynamic differences for each of these various classes of antimicrobial in the mouse model. However, mice treated with a combination of Mab166 and antibiotic (ciprofloxacin, tobramycin or ceftazidime) consistently exhibited greater survival rates compared with antibiotic treated mice over a 7 day period (FIG. 1B). Combination treated mice exhibited a 100% survival rate for the Mab166/Ciprofloxacin and Mab166/Tobramycin combinations, while the Mab166/Ceftazidime combination produced an 80% survival rate over this period of time. Overall, the combination therapy increased the survival rate by up to 100% compared to control mice and those treated with Mab166 alone. Compared to the next most efficacious treatment group, mice treated with antibiotic, the combination treatment improved survival by up to 40% over a 7 day observation period.

### Mab166/Antibiotic Combination Therapy Reduces Lung Injury.

The Mab166/tobramycin combination treatment produced a clear increase in mouse survival compared to the other treatment groups tested. To determine the basis of this improved survival, we first examined the extent of lung injury in each of the following treatment groups: untreated control, Mab166, tobramycin or Mab166/tobramycin. Three mice from each group were euthanized at 8 hours (just prior to death of the untreated control mice) and 24 hours (just prior to death of the Mab166 treated mice) post-infection. Surviving mice in the tobramycin and Mab166/tobramycin combination groups were euthanized 144 hours post-infection.

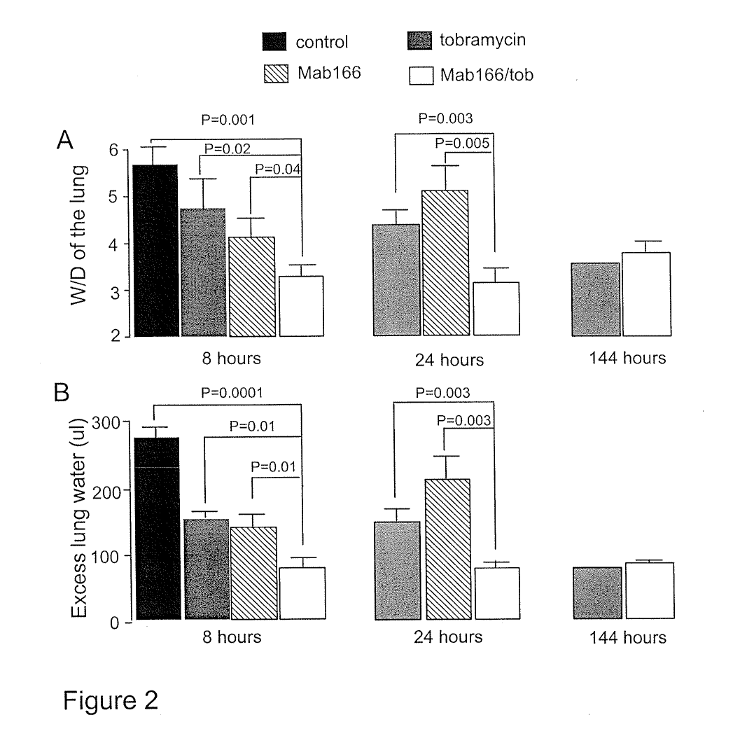

Wet/dry weight ratio and excess lung water was measured for mice from each group at each time point. At 8 hours post-infection, the untreated control group demonstrated the greatest lung injury, exhibiting greater wet/dry ratios and excess lung water compared to the Mab166, tobramycin or Mab166/tobramycin treated mice (FIGS. 2A and B). At this time point, the Mab166/tobramycin treatment group demonstrated significantly lower lung injury compared to the control, Mab166 only or tobramycin only treated groups (FIGS. 2A and B). At 24 hours post-infection a similar trend was observed; the combination therapy treated mice demonstrated significantly reduced lung injury compared to the Mab166 only or tobramycin only treated groups (FIGS. 2A and B). By 144 hours post-infection, only mice in the tobramycin and Mab166/tobramycin treated groups were available for analysis. At this time point, no significant difference in wet/dry ratio and excess lung water measurements was detected between these two treatment groups (FIGS. 2A and B).

### Mab166/Tobramycin Combination Therapy Protects Neutrophils in Lungs of Infected Animals.

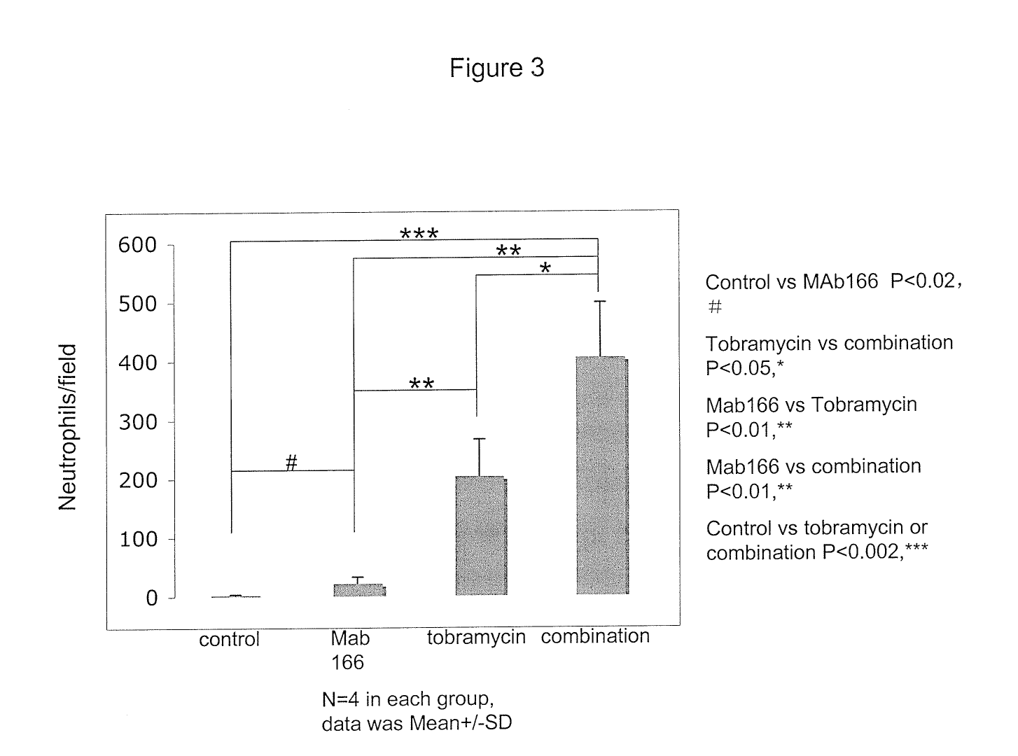

To investigate the mechanism by which the combination therapy resulted in better survival of infected mice, the number of neutrophils in the BAL (bronchoalveolar lavage, cytospin with H&E staining) fluid was analyzed in mice at 8 hours post-infection. Compared to Mab166 or tobramycin alone, the Mab166/tobramycin combination-treated animals exhibited a significantly higher (P<0.05 to P<0.01) number of intact neutrophils in the BAL fluid (FIG. 3) and better survival. Not to be bound by theory, these results are consistent with the hypothesis that TTSS inhibition by the antibody leads to protection of neutrophils in the lung. The fact that the combination treatment results in improved survival of the mice is surprising, as the lungs of these animals have the highest levels of inflammatory cells (neutrophils) at 8 hours post infection. High levels of cells may lead to inflammatory damage to the lungs and therefore reduced survival. The results suggest that combination therapy of antibiotic and antibody allows a controlled response to the infection and is therefore superior to mono therapy, especially where the P. aeruginosa is resistant or partially resistant to antibiotics.

Mab166/Tobramycin Combination Treatment Reduces Bacterial Numbers in the Lungs and Prevents Bacterial Dissemination into the Blood.

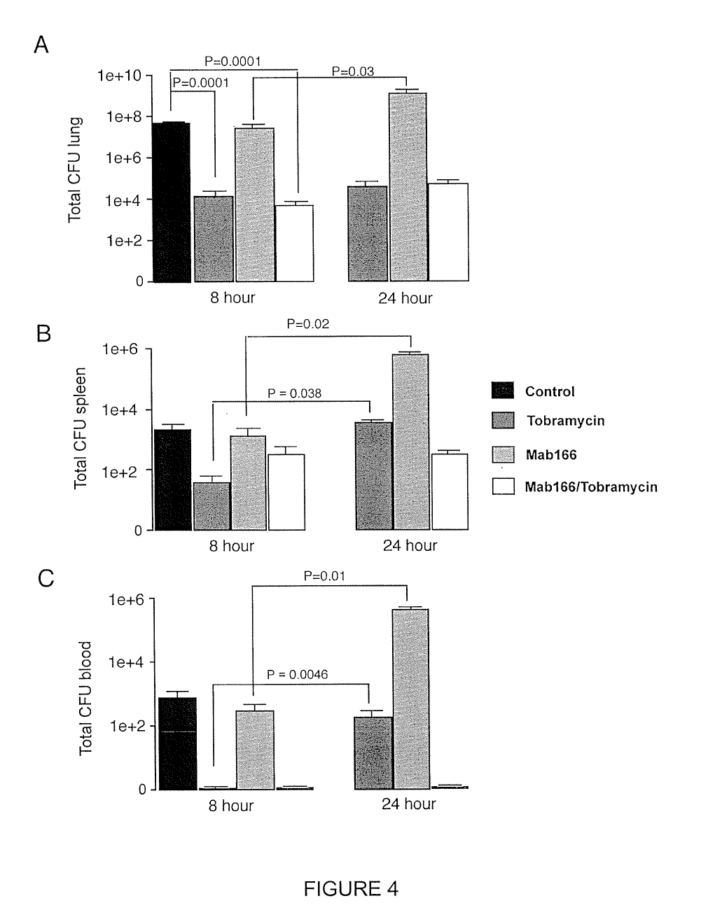

To further examine the basis of improved survival of mice administered the combination therapy, airway, blood and spleen samples were collected in parallel from mice in control, Mab166, tobramycin and Mab166/tobramycin treated groups. These samples were analyzed for bacterial CFU's as described in materials and methods. The total CFU's detected in the lungs of these mice was significantly lower for the Mab166/Tobramycin combination and tobramycin treated samples at both 8 and 24 hours post infection compared to the control mice (FIG. 4A). Administration of sub-optimal concentrations of Mab166 did not reduce bacterial numbers in the lungs of mice, in fact the numbers of CFU's increased significantly (p<0.03) from 8 to 24 hours in this group of mice. However, lung injury did not increase in this group of mice during this time period (FIG. 2), suggesting that while bacterial numbers increased, sufficient titer of Mab166 was present in the airways of these mice to prevent airway injury by the bacteria present.

Bacterial CFU's detected in the spleen were relatively similar (less than a log-fold difference in numbers) 8 hours post-infection across all 4 groups of mice. However, by 24 hours post infection, bacterial cell counts in the spleen had increased significantly in the Mab166 only treated group (P=0.02) and in the tobramycin treated group (P=0.038; FIG. 4B). Only the Mab166/tobramycin treated group exhibited a stable low number of bacterial CFU's in the spleen that did not increase over time (FIG. 4B). The observation that bacteria had disseminated to the spleen in the Mab166 and tobramycin treated groups was supported by the detection of significant (P=0.01, P=0.0046 respectively) increases in P. aeruginosa CFU's in the blood of mice from both these treatment groups 24 hour post infection (compared to respective 8 hour CFU's). The Mab166/tobramycin treated group demonstrated no evidence of bacterial colonies in blood at both 8 and 24 hours post infection, supporting that bacterial dissemination did not occur in animals in this treatment group. Subsequent analysis of Mab166 titer in the blood of Mab166/tobramycin treated mice demonstrated that antibody concentrations up to 4 μg per ml were present 7 days post administration. This titer was achieved from a single injection of Mab166 (300 μg ml⁻¹).

### Mab166/Tobramycin Combination Therapy Reduces Neutrophil Recruitment in Blood.

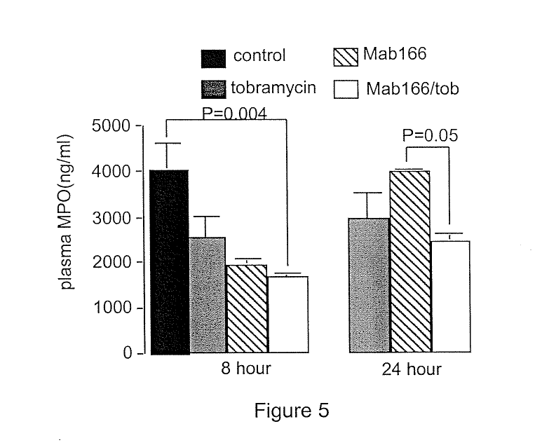

To confirm that the combination therapy resulted in lower bacterial numbers in blood and therefore less neutrophil recruitment and activity, we performed a Myeloperoxidase (MPO) assay on plasma collected 8 and 24 hours post-infection from the same mice used for bacterial CFU enumeration (FIG. 5). Compared to other groups, the Mab166/tobramycin combination treated animals exhibited significantly lower (P=0.04 and 0.05) MPO activity at both 8 and 24 hours, confirming the observation that bacterial numbers were lower in the blood of animals receiving this combination treatment.

Piperacillin and PcrV-Antibody Combination Therapy Against P. aeruginosa Airway Infection in Mice.

Anti-PcrV antibody was intravenously injected one hour before P. aeruginosa instillation. Mice were then anesthetized with avertin (250 mg/kg), prior to instillation of 1.5×10⁶ CFU PA103 into the trachea. Intraperitoneal piperacillin (1000 mg/kg, Q8H) injection commenced one hour after P. aeruginosa instillation and repeated until mice expired. Piperacillin was administered in combination with tazobactam (a pencillinase inhibitor), which is the standard practice for administration. In this example, “piperacillin” refers to piperacillin in combination with tazobactam. Anti-PcrV antibody Mab166 antibody was administered intravenously in the amount of 300 μg one hour before P. aeruginosa instillation. Four groups of animals (five animals per group) were analyzed: control animals that did not receive treatment; animals treated with PcrV antibody only; animals treated with piperacillin only; and animals treated with a combination of PcrV antibody plus piperacillin.

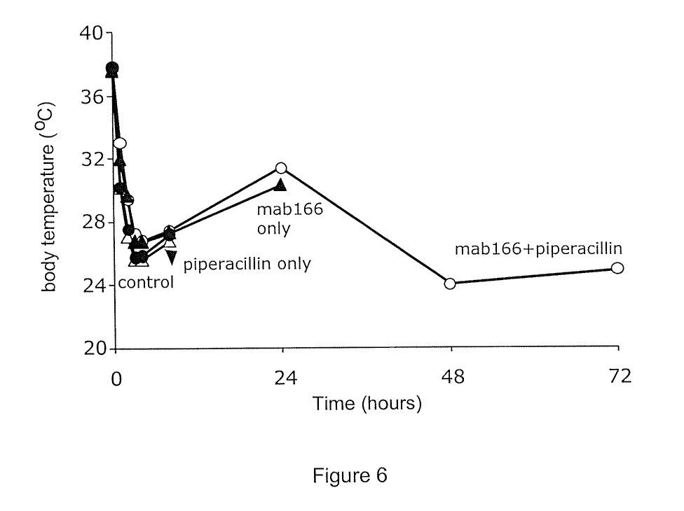

Body temperature declined dramatically in all four treatment groups, however, the Mab166 and Mab166 and piperacillin groups demonstrated a moderate recovery in body temperature prior to animal death (FIG. 6).

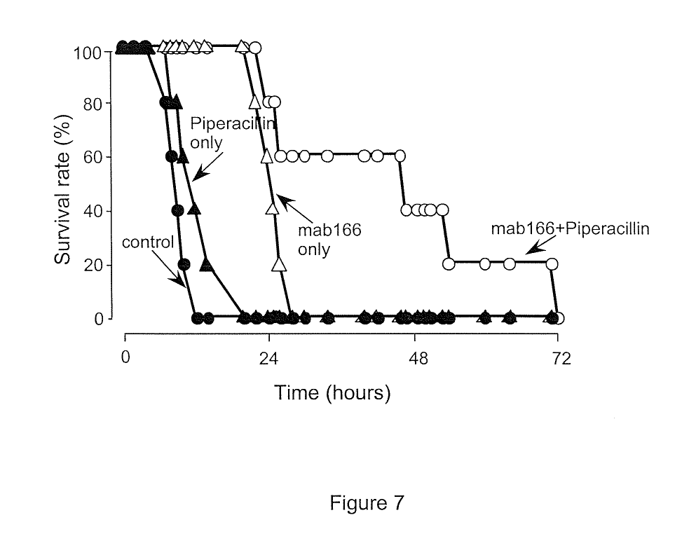

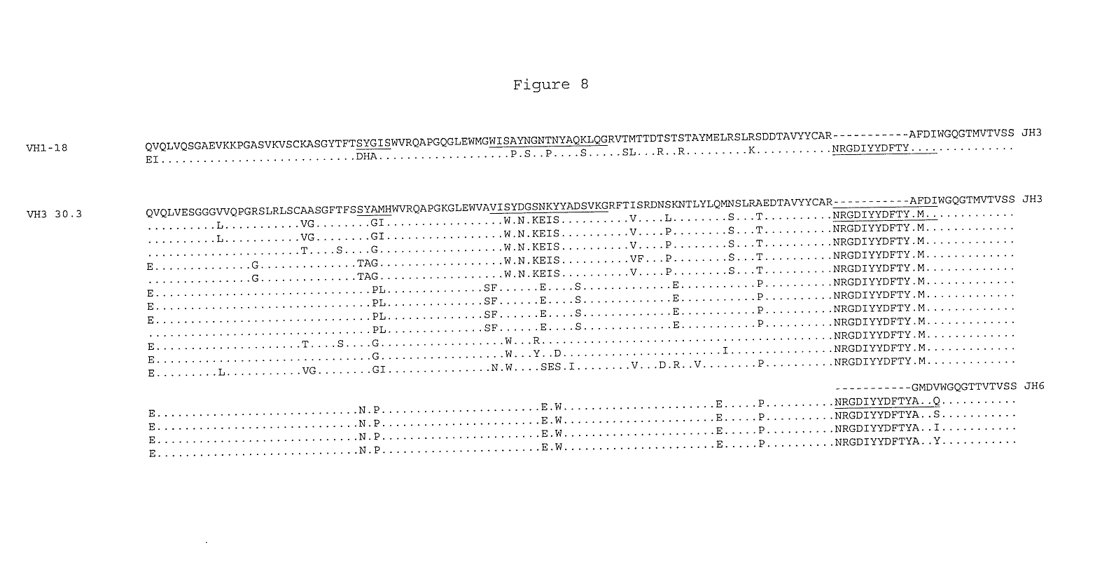

Survival curves (FIG. 7) demonstrated that the control mice in each experiment consistently died approximately 14 hours post-infection. Piperacillin treatment resulted in a slight increase in animal survival. Mab166 administration substantially prolonged mouse survival time compared to animals in the untreated control or piperacillin treated groups. However, mice in the Mab166 and piperacillin treated group exhibited the greatest survival.

### Summary

To examine the effects of a combination of antibiotics and PcrV antibody for treatment of P. aeruginosa infection, we employed a mouse model of acute infection using an overwhelming inoculum of 1.5×10⁶ CFU's, which is three times the lethal dose necessary to kill 90% of animals, of P. aeruginosa PA103. In addition, we used sub-optimal concentrations of Mab166 to determine if administration in combination with antimicrobials improved mouse survival.

Animals were divided into a control and three treatment groups: antibiotic, Mab166 or Mab166 and antibiotic combination. Control mice typically died approximately 14 hours post-inoculation. Animals treated with sub-optimal Mab166 therapy exhibited increased survival to approximately 36 hours, but this represented substantially poorer survival compared to antibiotic or Mab166 and antibiotic treated mice. Consistently, the combination of Mab166 and antibiotic (regardless of class) produced the greatest survival; in some cases all mice in the test group were alive 168 hours (7 days) post-infection. The combination therapy improved animal survival up to 40% over antibiotic treatment alone, indicating a synergistic effect between the antibiotic and the antibody in promotion of animal survival.

To determine the basis of enhanced survival in the combination therapy group, we compared several aspects of lung injury in the various groups of animals. Using wet/dry weight and excess lung water measurements as an indicator of lung injury, the combination treated group, compared to other treatment groups, exhibited significantly less lung injury over the initial 24 hours post-infection. By 144 hours post-infection, the lung injury present in both the antibiotic and combination treated groups was not significantly different suggesting that this aspect was not the key differential responsible for the observed survival differences between these two groups.

Patients with acute P. aeruginosa infection regularly succumb to multi organ failure due to dissemination of infection from the airways. ExoU⁺ P. aeruginosa strains are more commonly associated with acute invasive infections and are more frequently isolated from blood. To determine if spread of PA103 (ExoU-secreting) from the airways played a role in survival differences of the control and treatment groups, bacteria in the lungs, blood and spleen were enumerated. In the control and Mab166 only treated groups, bacterial numbers were relatively equivalent and significantly higher 8 hours post-infection than those detected in the antibiotic only or combination treated animals. Despite the large number of bacterial cells present, the Mab166 treated group exhibited significantly less lung injury 8 hours post infection, suggesting that protection by the antibody against epithelial damage and prevention of bacteremia were the key differentials in animal survival between the control and Mab166 treated groups at the outset of infection. The Mab166 dose administered to these animals in this study was sub-optimal, presumably resulting in a saturation effect and an inability of the antibody to neutralize the cytotoxic effect of all P. aeruginosa cells in a proliferating population (bacterial CFU's in the lung and lung injury had increased significantly in this treatment group 24 h post-infection). Bacterial numbers in the blood and spleen also increased dramatically in the Mab166 treated group 24 hours post-infection. Together these observations suggest that while sub-optimal concentration of Mab166 reduces lung injury at the outset of infection, it is insufficient to prevent long-term airway epithelial damage and dissemination of the infection to other organs in the case of an overwhelming infection with an ExoU secreting strain.

For the tobramycin treated group, greater lung injury was observed in this group at the outset of infection compared to the combination treated group. Additionally, while bacterial cell numbers remained stable in the airways of animals in this treatment group, they increased significantly in the blood and spleen over the initial 24 hours post-infection. In this case it appears that while the antibiotic can reduce bacterial proliferation locally at the point of infection, it is not sufficient to prevent dissemination of bacterial cells to discreet organs and the blood. In comparison, the Mab166 and antibiotic combination treated animals consistently exhibited the lowest lung injury, stable numbers of bacteria in the airways and no evidence of CFU's in the blood or spleen. MPO measurements support the assertion that bacterial numbers were lowest in the blood of animals who received the combination therapy. Therefore, prevention of acute invasive infection appears to be the crucial differential that contributes to improved animal survival in the combination treated animals.

The results using piperacillin show that high doses of piperacillin increased survival minimally, while Mab166 treatment significantly increased survival time. The combination therapy (Mab166 and piperacillin) significantly increased mouse survival time. Conventional antibiotic resistance testing demonstrated that PA103 was sensitive to piperacillin, however, as assessed from mouse death, in vivo resistance of this strain appears to be greater. This is putatively due to altered pharmacokinetics or pharmacodynamics of this antibiotic in a mouse model. However, similar to experiments with other more potent antibiotics, the protective effect of the combination of Mab166 and piperacillin administration exhibited enhanced superiority compared to each of these therapies administered individually.

P. aeruginosa is a significant clinical problem. Blood stream infections by this species are particularly problematic and have a mortality rate that ranges from 18-62% (Vidal et al., Arch. of Int. Med. 156:2121-2126, 1996). The studies described in these examples show that the combination of Mab166 and antibiotic therapy results in improved outcome in a murine model of overwhelming acute infection with a potent ExoU-secreting P. aeruginosa strain. These results indicate that the synergistic effects of the treatment methods of the invention will improve outcome in patients with acute airway infection and prevent dissemination of infection.

### Example 2

### Identification of Engineered Human Anti-PcrV Fab Molecules for Use in the Invention

Epitope-focused engineered human antibody Fab libraries were generated as described in US patent application 20050255552. V-segment sequences derived from repertoires of human immunoglobulin sequences were cloned upstream of a selected CDR3-FR4 sequence for each of the heavy and light chains.

For heavy-chain repertoires, the CDRH3 comprises a D-segment derived sequence (NRGDIYYDFTY) from a previously identified anti-PcrV monoclonal antibody (Mab166; Frank et al 2002 J. Infectious Dis. 186: 64-73) which constitutes a binding specificity determinant. The sequence of the complete CDRH3-FR4 sequence for the heavy chain repertoires is shown below.

For VH1 library 1015, the CDR3-FR4 combination used was:

CDR3
NRGDIYYDFTYAFDIWGQGTMVTVSS (FR4 = JH3)

For VH3 libraries, the CDR3-FR4 combination used differed by a single amino acid in CDRH3:

CDR3
NRGDIYYDFTYAMDIWGQGTMVTVSS (FR4 = JH3)

For light-chain repertoires, human Vkappa or Vlambda sequences comprising FR1-CDRL1-FR2-CDRL2-FR3 were inserted upstream of selected CDRL3-FR4 sequences. The CDRL3 comprises a binding specificity determinant from Mab166 light-chain with the sequence FWXTP (where X may be S or G). For Vkappa libraries, the C-terminal residues of CDRL3 and FR4 were contributed by the human germ-line JK2 sequence YTFGQGTKLEIK (JK2 residues within CDRL3 are underlined). For Vlambda libraries, the FR4 region was contributed by JL2 germ-line sequence FGGGTKLTVL. The JL2 germline sequence is identical to the JL3 sequence.

In some cases cassette libraries were constructed as described in US patent application 20060134098 (library 1070). For library 1080, full-length lambda chains were screened in combination with VH cassette libraries.

Heavy and light chain polypeptides were expressed as mature proteins, i.e., without a signal peptide, and secreted in E. coli cells that express a mutant SecY gene as described in US patent application 20070020685. The peptides therefore were expressed with an N-terminal methionine. Binding of recombinant Fabs to PcrV was identified by a filter-binding assay using nitrocellulose filters coated with GST-PcrV fusion protein as described in US patent application 20050255552. Binding activity was confirmed by antigen ELISA using plates coated with GST-PcrV and affinities were determined by biolayer interferometry using a ForteBio Octet biosensor.

The sequences of the V-regions of exemplary high-affinity anti-PcrV antibodies are shown in FIG. 8 and FIG. 9.

Each of the Fabs has high affinity for PcrV. Several Fabs were identified with affinities at least equivalent to Mab166 Fab (approximately 1.4 nM) determined by biolayer interferometry using a ForteBio (Mountain View, Calif.) Octet biosensor.

V_{H} and V_{L} regions identified as described can be used in various combinations. For example, a V_{K} light chain SEQ ID NO:12 supports high affinity binding to PcrV in combination with either a V_{H} comprising SEQ ID NO:11, or a V_{H} comprising SEQ ID NO:3.

The 1070-9E antibody is an example of a high affinity antibody derived by V-region cassette exchange using methods described in US Patent Application Publication No. 20060134098. To isolate this antibody, 4 V-region replacement cassettes were constructed:

1) heavy chain front-end cassette (consisting of human VH3 FR1-CDR1-FR2 sequences)
2) heavy chain middle cassette (consisting of human VH3 FR2-CDR2-FR3 sequences)
1) light chain front-end cassette (consisting of human VK1 FR1-CDR1-FR2 sequences)
2) light chain middle cassette (consisting of human VK1 FR2-CDR2-FR3 sequences).
Each cassette was assembled with additional V-region sequences from Mab166 and the selected CDR3-FR4 region and expressed as Fab fragments in E. coli TOP10 cells transformed with a plasmid over-expressing a mutant SecY gene to allow secretion of signal-less Fabs. Cassette Fab libraries were then screened on GST-PcrV coated filters to identify PcrV binders. Selected sequences from Fabs supporting PcrV binding were then recombined and re-screened to identify fully-human V-segments supporting high-affinity binding to PcrV.

Fab 1070-9E, isolated by cassette recombination, has an affinity for recombinant PcrV of 1.48 nM, determined by biolayer interferometry.

High-affinity anti-PcrV Fabs are also potent antagonists of the P. aeruginosa Type

III Secretion system and inhibit P. aeruginosa exotoxin-mediated killing of P3-X63 Ag8 myeloma cells by P. aeruginosa strain PA103 in a cell-based cytotoxicity assay.

### Example 3

### PEGylated Humaneered Fab′

In this example, a Fab′ consisting of a human Fd′ heavy chain of the IgG1 sub-class and human kappa light chain linked by an inter-chain disulfide bond involving the C-terminal cysteine of the kappa chain and the cysteine residue C227 of the heavy chain (numbering sequentially from the N-terminus of the mature protein) was PEGylated. The recombinant Fd′ heavy chain contains the IgG1 CH1 domain and the IgG1 hinge region including two cysteine residues which are available after reduction for conjugation to maleimide groups. Thus the expressed antibody protein is a disulfide-linked heterodimer of Fd′ heavy chain and a kappa light chain, containing a total of 452 amino acids.

To generate an immunoconjugate with a reduced rate of in vivo clearance and thus an improved pharmacokinetic profile, the Fab′ is conjugated to polyethylene glycol (PEG). In di-PEGylated Fab′, each molecule of Fab′ is conjugated to two long-chain PEG molecules by site-specific attachment at the hinge region exploiting the two available reactive thiols on the hinge cysteine residues and a maleimide derivatized PEG, methoxy-polyethylene glycol maleimide (mPEG-mal). The mPEG-mal molecules are conjugated via thioether linkages between the maleimide moiety and the hinge cysteine residues.

To generate di-PEGylated Fab′, mPEG-mal with average molecular weight of 30 kD was obtained from NOF Corporation. The Fab′, which was expressed and secreted from E. coli, was prepared at a concentration of 4 mg/ml in sodium citrate buffer pH 6.5 with 2 mM EDTA. Reducing agent (10 mM MEA at pH 6.5) was added for 30 minutes at room temperature and the reaction mixture was immediately desalted using a Zeba Desalt column (Pierce) pre-equilibrated with 10 mM glycine (pH 3) and 2 mM EDTA. mPEG-mal was added for 1 hour at room temperature and di-PEGylated Fab′ was separated from other PEGylated species and from unreacted Fab′ using a HiTrap SP sepharose column on an Akta purification system from GE Healthcare.

The exemplary di-PEGylated Fab′ PEGylated in this example binds with high affinity to PcrV (affinity of 0.6 nM determined by surface plasmon resonance analysis) and is a potent antagonist of the P. aeruginosa Type III Secretion System.

### Example 4

### Cytotoxicity Assay for Detection of Antibodies and Fab Fragments for Use in the Invention that have Potent Neutralization Activity Against the P. aeruginosa Type III Secretion System

A TTSS-dependent cytotoxicity assay was established using P3-X63-Ag8 (X63) mouse myeloma cells (ATCC) as the target. Cells were cultured in RPMI 1640 (Media Tech) with 10% FBS (Hyclone). About 10⁵ cells were infected with P. aeruginosa strain PA103 at a multiplicity of infection (MOI) of 10 in a volume of 0.1 ml culture medium in wells of a 96-well plate in the presence of Fab. Prior to addition of Fab and mammalian cells, PA103 was grown in MinS medium (Hauser, et al. (1998) Infect Immun. 66:1413-1420) to induce expression of the TTSS. After incubation for three hours at 37° C. with 5% CO2, with various concentrations of anti-PcrV Fab, cells were transferred to 12×75 mm flow-cytometry tubes and stained with propidium iodide (Sigma) according to the manufacturer's instructions. The proportion of permeabilized cells was quantified by flow cytometry using a FACS Caliber flow cytometer. Data were analyzed using Prism4 software (Graphpad). (Cytotoxicity was normalized to dead cells in untreated samples). For comparison of the potency of different Fabs, mean concentrations required for 50% inhibition (IC₅₀) were obtained from at least 3 independent assays. Results for several exemplary Fabs are shown in Table 3 below.

TABLE 3
Potency of Fabs in cytotoxicity assay

| Fab | IC₅₀ (nM) |
|---|---|
| Mab166 Fab | 53.0 |
| SEQ ID NOs: 13, 4 | 20.0 |
| SEQ ID NOs: 13, 37 | 12.0 |
| SEQ ID NOs: 5, 6 | 50.2 |
| SEQ ID NOs: 13, 10 | 25.5 |
| SEQ ID NOs 3, 4 | 35.1 |
| SEQ ID NOs. 24, 26 | 25.5 |
| SEQ ID NOs. 35. 36 | 61.4 |

Each of the Fabs tested shows potent neutralization of the TTSS and protection of mammalian cells from cytotoxicity.

Several Fabs are more potent in this assay than Mab166 Fab. Thus, anti-PcrV antibodies of the invention typically show enhanced potency relative to Mab166 Fab.

### Example 5

### A Humaneered Antibody Shows In Vivo Efficacy Using a Mouse Model of Pneumonia

Experiments were performed in vivo using humaneered Fabs to evaluate the effects of the antibodies in a mouse model of pneumonia. Fab 1A8 has a human VH3 sub-class heavy chain, containing the first constant domain of human IgG1, and a human VKI sub-class kappa light chain. The affinity of Fab 1A8 as determined by Biacore is 0.6 nM. Fab 1A8 binds to PcrV with approximately two-fold higher affinity than Mab166 Fab.

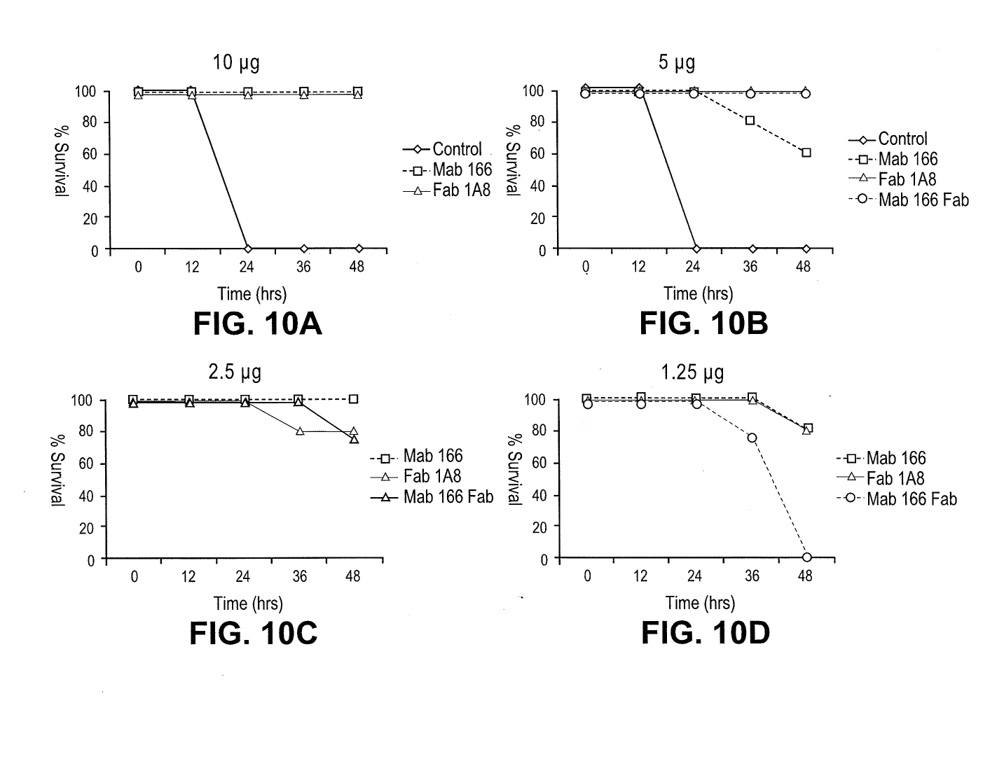

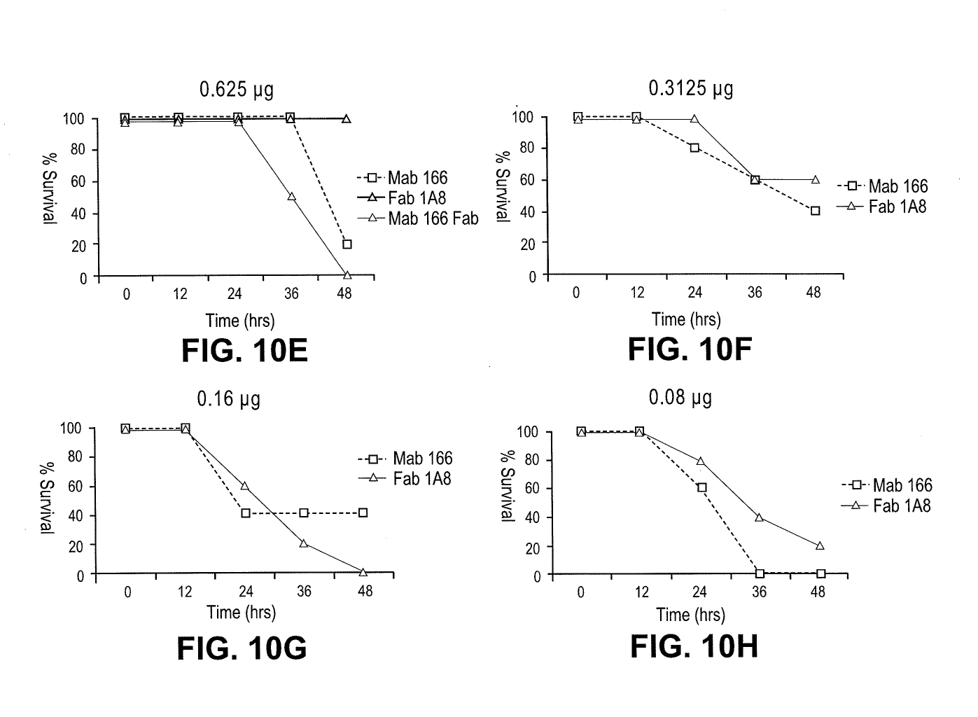

An acute lethality model of Pseudomonas pneumonia was used to assess the in vivo efficacy of Fab 1A8 in comparison with Mab166. P. aeruginosa strain PA103 was instilled directly into the lungs of mice at a dose of 1.5×10⁶ cfu/mouse by intratracheal administration, an inoculum shown previously to be sufficient to lead to lethality in 100% of the animals (3×LD₉₀) (Sawa et al., Nat. Med. 5:392-8, 1999). Survival and body temperature were monitored for 48 hours and surviving mice at this time point were sacrificed for determination of bacterial counts in the lungs. The survival data (FIG. 10) indicated that both the human Fab 1A8 and the murine Fab were able to prevent lethality caused by the highly cytotoxic PA103 strain. Control mice infected with PA103 and treated with an irrelevant control Fab, were all dead within 24 hours of inoculation. Treatment of mice with 10 μg Mab166 or Fab 1A8 led to the survival of 100% of the mice at 48 hours. Since Fab 1A8 lacks the antibody Fc-region, antibody effector functions are not required for prevention of lethality. Fab 1A8 was significantly more potent than Mab166 Fab in prevention of lethality. Fab 1A8 provided significant protection from lethality at doses of 1.25 μg and 0.625 μg/mouse, doses at which mouse Mab166 Fab-treated animals showed 100% mortality (P<0.05 for differences between Fab 1A8 and Mab166 Fab at 2.5 μg, 1.25 μg and 0.625 μg doses). The activity of Fab 1A8 is comparable to that of Mab166 IgG in prevention of lethality.

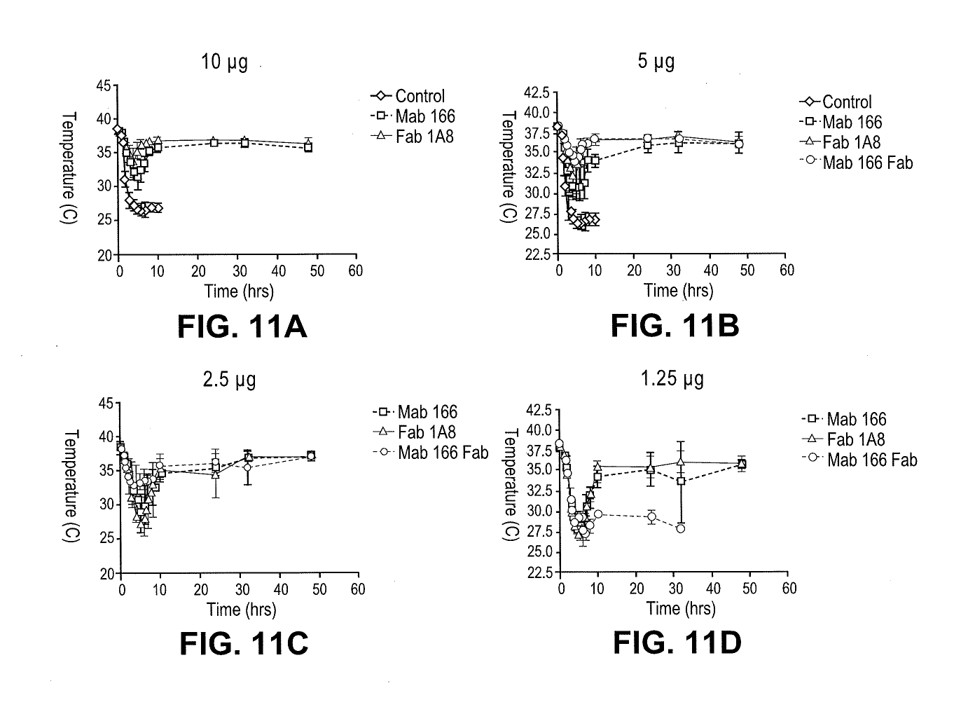

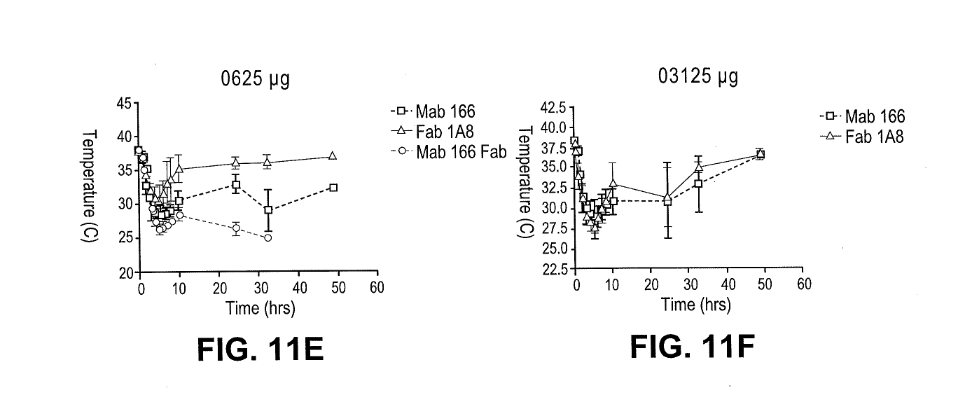

Fab 1A8 was also effective in inducing recovery of body temperature, indicative of protection from sepsis (FIG. 11). Untreated mice infected with PA103 showed a rapid drop in body temperature within the first few hours of infection. Recovery of body temperature within 12-24 hours in the antibody-treated groups correlated with subsequent survival. Doses as low as 1.25 μg/mouse of Fab 1A8 or Mab166 led to rapid recovery of body temperature and prevented lethality in at least 80% of mice. However, this dose of mouse Mab166 Fab fragment was insufficient to allow body temperature recovery and all mice in this group were dead at 48 hours post-infection.

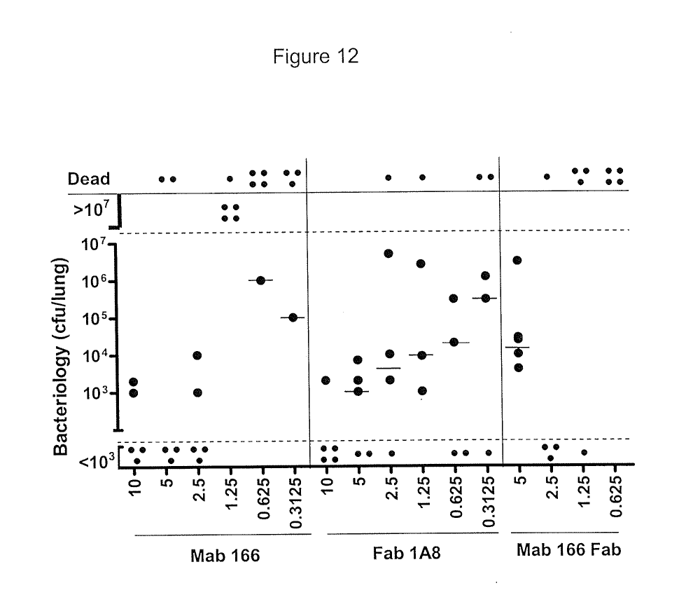

Surviving mice at 48 hours post-challenge were also analyzed for the presence of residual P. aeruginosa in the lungs. Both Mab166 and the Fab 1A8 fragments analyzed stimulated significant clearance of bacteria (FIG. 12). After 48 hours, the bacterial counts were reduced at least 1000-fold from the infectious dose of 1.5×10⁶ cfu/mouse in all mice treated with 10 μg Fab 1A8.80% of mice treated with this dose of Fab 1A8 showed no detectable P. aeruginosa in the lungs after 48 hours. Higher residual bacterial counts were detected in mice treated with mouse Mab166 Fab. Human Fab 1A8 has comparable potency to the whole IgG Mab166 in this analysis indicating that Fc-effector functions do not contribute significantly to the ability of the antibody to stimulate bacterial clearance

A second humaneered Fab that has the Mab166 minimal essential binding specificity determinant was also evaluated in vivo using a mouse model of pneumonia. Female Balb/c mice (approximately 20 g in weight; Charles River) were inoculated with 1×10⁶ P. aeruginosa strain PA103 by intra-tracheal administration. Prior to inoculation, PA103 bacteria were grown overnight in YPT broth at 37° C., diluted 1:5 in fresh medium and grown for two hours at 37° C. until they reached exponential phase. The culture was centrifuged at room temperature for ten minutes at 2000×g and the pellet resuspended in ˜8 mL phosphate buffered saline (PBS). Bacteria were quantified by absorbance at 600 nm and bacterial colony-forming units verified by colony growth on tryptic soy (TS) agar plates (Teknova, Half Moon Bay, Calif.). Antibody Fab 2 fragment was premixed with bacteria immediately prior to intratracheal instillation. Infected mice were monitored for body temperature (rectal temperatures) and survival for 48 hours.

Control mice treated only with saline solution showed 100% mortality within 24 hours of bacterial inoculation. Mice treated with 10 μg Fab 2 showed complete protection from lethality; 100% of the Fab-treated mice survived at 48 hours.

This example thus demonstrates that humaneered antibodies of the invention exhibit potent in vivo activity against P. aeruginosa. The Fabs are more potent than a parent Mab166 Fab in vivo.

### Example 6

### Evaluation of a Humaneered Fab for Immunogenicity in Human

An engineered antibody PEGylated Fab′ fragment was evaluated for safety, immunogenicity and plasma/serum half-life in human subjects. Subjects received one dose by intravenous (i.v.) injection at 1, 3, or 10 mg/kg.

The engineered antibody was well tolerated at all dose levels. The concentration of drug in the plasma was measured by ELISA using the PcrV antigen immobilized onto a microtiter plate. GST-PcrV was immobilized onto a microtiter plate overnight at 4° C. The plate was washed and all unadsorbed sites blocked with the addition of block/diluent buffer for at least 60 minutes. After washing the plate, analytes were dispensed onto the pre-coated microtiter plate and incubated for at least 60 minutes. The plate was washed and a solution containing a biotinylated antibody specific to the engineered Fab was added for 45 minutes. The plate was washed and a HRP-conjugate solution added for 30 minutes. After the final wash step, a tetramethylbenzidine (TMB) peroxidase substrate solution was added and incubated for approximately 6 minutes. The reaction was stopped with a phosphoric acid solution. Color develops in proportion to the amount of PEGylated Fab present. Plates were read on a plate reader using two filters (450 nm for detection and 620 nm for background). Concentrations were determined on a standard curve obtained by plotting optical density (OD) versus concentration. The calibration curve was generated using a four-parameter logistic fit. The range for this method in human serum is from 0.200 to 12.8 ng/mL in 1% serum (20.0 ng/mL to 1280 ng/mL in 100% serum).

Pharmacokinetic Profile of Engineered Antibody in Human Subjects

| PK Parameter | Units | n | 1.0 mg/kg Cohort 1 | | 3.0 mg/kg Cohort 2 | | 10.0 mg/kg Cohort 3 | |
|---|---|---|---|---|---|---|---|---|
| AUC(0-t) | ng*hr/mL | 4 | 10440826 | (1137824) | 33973664 | (2930669) | 120424224 | (17896301) |
| AUC(0-∞) | ng*hr/mL | 4 | 10737696 | (1235316) | 34856666 | (3216244) | 124429066 | (19035293) |
| % Extrap | (%) | 4 | 2.72 | (0.886) | 2.49 | (0.935) | 3.17 | (0.461) |
| Cmax | (ng/mL) | 4 | 29334 | (2039) | 93533 | (10738) | 347287 | (64571) |
| T½ | (hr) | 4 | 341 | (38.5) | 310 | (37.9) | 338 | (14.1) |
| CL | (L/hr) | 4 | 0.00693 | (0.000823) | 0.00556 | (0.000573) | 0.00473 | (0.00112) |
| Vz | (L) | 4 | 3.44 | (0.730) | 2.50 | (0.504) | 2.31 | (0.588) |

The engineered antibody had a terminal plasma half-life of approximately 14 days.

The presence of anti-drug antibodies, i.e., antibodies generated to the humaneered antibody, was tested at: pre-infusion, day 8, day 15 day 29 and day 70 post infusion. Anti-drug antibodies were measured using an electrochemiluminescent assay (ECLA). Positive controls and negative control serum were diluted 1:25 with diluent buffer. The controls were further diluted 1:2 by the addition of an equal volume of 0.8% acetic acid (resulting in 2× solutions) and then incubated at ambient temperature for approximately 15 minutes. Samples were then diluted an additional 1:2 with Label Master Mix (Antibody-Biotin and Antibody-SulfoTag at 0.5 μg/mL final working concentrations) resulting in a final 1:100 dilution. All controls were then incubated for one hour at room temperature with gentle shaking. The Streptavidin-coated standard MA2400 96-well microtiter plate was blocked by adding diluent buffer for 60 minutes. Diluent buffer was removed from plate wells by aspiration and controls were added to the plate and incubated for 60 minutes. The plate was aspirated and washed, and 1× MesoScaleDiscovery® (MSD) Read Buffer T with surfactant was added. The plates were read on an MSD electrochemiluminescence detector within 1 minute. Intensity of relative light units (RLU) produced are in proportion to the amount of anti-drug antibody present.

No anti-drug antibodies were detected at any time point. This example thus demonstrates that there was no detectable immunogenicity of the humaneered antibody in humans.

The following provides an exemplary listing of anti-PcrV antibody V-regions for use in the invention:

### Exemplary Anti-PcrV V-Regions

SEQ ID NO: 1 Vh (VH1)
EIQLVQSGAEVKKPGASVKVSCKASGYTFTDHAISWVRQAPGQGLEW
MGWISPYSGNPNYAQSLQGRVSLTTDRSTRTAYMELRSLKSDDTAVY
YCARNRGDIYYDFTYAFDIWGQGTMVTVSS
SEQ ID NO: 2 VkI
DIQMTQSPSSVSASVGDRVTITCRASQGISSWLAWYQQKPGRAPKLL
IYAASSLQSGVPSRFSGSGSGTGFTLTISSLQPEDVATYYCQQFWST
PYTFGQGTKLEIK
SEQ ID NO: 3 Vh
QVQLVESGGGVVQPGGSLRLSCAASGFTFSTAGMHWVRQAPGKGLEW
VAVIWYNGKEISYADSVKGRFTVSRDNPKNTLYLQMSSLRTEDTAVY
YCARNRGDIYYDFTYAMDIWGQGTMVTVSS
SEQ ID NO: 4 VkI
DIQMTQSPSSLSASVGDRVTITCRASQSISRWVAWYQQRPGKAPNLL
IYDASSLKSGVPSRFSGSGSGTEFTLTISSLQPEDIATYYCQQFWST
PYTFGQGTKLEIK
SEQ ID NO: 5 Vh
QVQLVESGGGVVQPGRSLRLSCTASGFSFSSYGMHWVRQAPGKGLEW
VAVIWYNGKEISYADSVKGRFTVSRDNPKNTLYLQMSSLRTEDTAVY
YCARNRGDIYYDFTYAMDIWGQGTMVTVSS
SEQ ID NO: 6 VkI
AIQLTQSPSFLSASVGDRVTITCRASQGISTYLAWYQQKPGKAPKLL
IYAASSLQSGVPSRFSGSGSGTDFTLTISSLQPEDVATYYCQQFWST
PYTFGQGTKLEIK
SEQ ID NO: 7 Vh
QVQLVESGGGLVQPGRSLRLSCVGSGFTFSSYGIHWVRQAPGKGLEW
VAVIWYNGKEISYADSVKGRFTVSRDNLKNTLYLQMSSLRTEDTAVY
YCARNRGDIYYDFTYAMDIWGQGTMVTVSS
SEQ ID NO: 8 VkI
DIQMTQSPSFLSASVGDRVTITCRASQGISTYLAWYQQKRGKAPKLL
ISAASSLQSGVPSRFSGSVSGTDFTLTISSLQSEDFAVYYCQQFWST
PYTFGQGTKLEIK
SEQ ID NO: 9 Vh
QVQLVESGGGLVQPGRSLRLSCVGSGFTFSSYGIHWVRQAPGKGLEW
VAVIWYNGKEISYADSVKGRFTVSRDNPKNTLYLQMSSLRTEDTAVY
YCARNRGDIYYDFTYAMDIWGQGTMVTVSS
SEQ ID NO: 10 VkI
DIQLTQSPSFLSASVGDRVTITCRASQGISTYLAWYQQKPGKAPKLL
IYDASALQSGVPSRFSGSGSGTEFTLTISSLQPEDVATYYCQQFWST
PYTFGQGTKLEIK
SEQ ID NO: 11 Vh
EVQLVESGGGVVQPGGSLRLSCAASGFTFSTAGMHWVRQAPGKGLEW
VAVIWYNGKEISYADSVKGRFTVFRDNPKNTLYLQMSSLRTEDTAVY
YCARNRGDIYYDFTYAMDIWGQGTMVTVSS
SEQ ID NO: 12 VkI
DIQMTQSPSSLSASVGDRVTITCRASQSISRWVAWYQQRPGKAPNLL
IYDASSLKSGVPSRFSGSGSGTEFTLTISSLQPEDIATYYCQQFWST
PYTFGQGTKLEIK
SEQ ID NO: 13 Vh
QVQLVESGGGVVQPGRSLRLSCAASGFTFSSYPLHWVRQAPGKGLEW
VSFISYDGSEKYYASSVKGRFTISRDNSENTLYLQMNSLRPEDTAVY
YCARNRGDIYYDFTYAMDIWGQGTMVTVSS
SEQ ID NO: 14 Vk
DIQLTQSPSFLSASVGDRVTITCRASQGISTYLAWYQQKPGKAPKLL
IYDASALQSGVPSRFSGSGSGTEFTLTISSLQPEDVATYYCQQFWST
PYTFGQGTKLEIK
SEQ ID NO: 15 Vh
EVQLVESGGGVVQPGRSLRLSCTASGFSFSSYGMHWVRQAPGKGLEW
VAVIWYDGRNKYYADSVKGRFTISRDNSKNTLYLQMNSLRAEDTAVY
YCARNRGDIYYDFTYAMDIWGQGTMVTVSS
SEQ ID NO: 16 VkIII
EIVLTQFPGTLSLSPGERATLSCRASQNVGSAYLAWYQQKPGQAPRL
LIYGASRRAPGIPDRFSGSGSGTDFTLTINRLEPEDFAVYYCQQFWS
TPYTFGQGTKLEIK
SEQ ID NO: 17 Vh
EVQLVESGGGVVQPGRSLRLSCAASGFTFSSYGMHWVRQAPGKGLEW
VAVIWYDGYNKDYADSVKGRFTISRDNSKNTLYLQINSLRAEDTAVY
YCARNRGDIYYDFTYAMDIWGQGTMVTVSS
SEQ ID NO: 18 VkIII
EIVMTQSPATLSVSPGERATLSCRASQSVSSNLAWYQQKPGQAPRLL
IYGASTRATGIPARFSGSGSGTEFTLTISSLQSEDFAVYYCQQFWST
PYTFGQGTKLEIK
SEQ ID NO: 19 Vh
EVQLVESGGGVVQPGRSLRLSCAASGFTFSSYPLHWVRQAPGKGLEW
VSFISYDGSEKYYASSVKGRFTISRDNSENTLYLQMNSLRPEDTAVY
YCARNRGDIYYDFTYAMDIWGQGTMVTVSS
SEQ ID NO: 20 VkIII
EIVMTQSPATLSVSPGERATLSCRASQSVSSNLAWYQQKPGQAPRLL
FYAASTRATGIPARFSGSGSGTEFTLTISSLQSEDFAVYYCQQFWST
PYTFGQGTKLEIK
SEQ ID NO: 21 Vh
EVQLVESGGGLVQPGRSLRLSCVGSGFTFSSYGIHWVRQAPGKGLEW
VANIWYDGSSESYIDSVKGRFTVSRDDSRNTVYLQMNSLRPEDTAVY
YCARNRGDIYYDFTYAMDIWGQGTMVTVSS
SEQ ID NO: 22 VkIII
EIVMTQSPATLSVSPGERATLSCRASQSVSSNLAWYQQKPGQAPRLL
IYGASTRATGIPARFSGSGSGTEFTLTISSLQSEDFAVYYCQQFWST
PYTFGQGTKLEIK
SEQ ID NO: 23 VH
EVQLVESGGGVVQPGRSLRLSCAASGFTFSNYPMHWVRQAPGKGLEW
VAVISYDGSEKWYADSVKGRFTISRDNSKNTLYLEMNSLRPEDTAVY
YCARNRGDIYYDFTYAMDQWGQGTTVTVSS
SEQ ID NO: 24 VK
DIQLTQSPSTLSASVGDSVTITCRASEGVDRWLAWYQQKPGRAPKLL
IYDASTLQSGVPSRFSGSGSGTEFSLTISSLQPDDVATYYCQHFWGT
PYTFGQGTKLEIK
SEQ ID NO: 25 VH
EVQLVESGGGVVQPGRSLRLSCAASGFTFSNYPMHWVRQAPGKGLEW
VAVISYDGSEKWYADSVKGRFTISRDNSKNTLYLEMNSLRPEDTAVY
YCARNRGDIYYDFTYAMDSWGQGTTVTVSS
SEQ ID NO: 24 VK
DIQLTQSPSTLSASVGDSVTITCRASEGVDRWLAWYQQKPGRAPKLL
IYDASTLQSGVPSRFSGSGSGTEFSLTISSLQPDDVATYYCQHFWGT
PYTFGQGTKLEIK
SEQ ID NO: 26 VH
EVQLVESGGGVVQPGRSLRLSCAASGFTFSNYPMHWVRQAPGKGLEW
VAVISYDGSEKWYADSVKGRFTISRDNSKNTLYLEMNSLRPEDTAVY
YCARNRGDIYYDFTYAMDIWGQGTTVTVSS
SEQ ID NO: 24 VK
DIQLTQSPSTLSASVGDSVTITCRASEGVDRWLAWYQQKPGRAPKLL
IYDASTLQSGVPSRFSGSGSGTEFSLTISSLQPDDVATYYCQHFWGT
PYTFGQGTKLEIK
SEQ ID NO: 35 VH
EVQLVESGGGVVQPGRSLRLSCAASGFTFSNYPMHWVRQAPGKGLEW
VAVISYDGSEKWYADSVKGRFTISRDNSKNTLYLEMNSLRPEDTAVY
YCARNRGDIYYDFTYAMDYWGQGTTVTVSS
SEQ ID NO: 36 VK
DIQLTQSPSTLSASVGDSVTITCRASEGVDRWLAWYQQKPGRAPKLL
IYDASTLQSGVPSRFSGSGSGTEFSLTISSLQPDDVATYYCQHFWST
PYTFGQGTKLEIK

V-regions of Exemplary Antibodies with Lambda Light Chain

SEQ ID NO: 27 Vh
EVQLVESGGGVVQPGRSLRLSCAASGFTFSSYPLHWVRQAPGKGLEW
VSFISYDGSEKYYASSVKGRFTISRDNSENTLYLQMNSLRPEDTAVY
YCARNRGDIYYDFTYAMDIWGQGTMVTVSS
SEQ ID no: 28 Vl
QSALTQPASVSGSPGQSITISCTGTSSDYVSWYQQHPGKAPKLIIYD
VTNRPSGVPDRFSGSKSGNTASLTISGLQAEDEADYYCQHFWSTPYT
FGGGTKLTVL
SEQ ID NO: 29 Vh
EVQLVESGGGVVQPGRSLRLSCAASGFTFSSYPLHWVRQAPGKGLEW
VSFISYDGSEKYYASSVKGRFTISRDNSENTLYLQMNSLRPEDTAVY
YCARNRGDIYYDFTYAMDIWGQGTMVTVSS
SEQ ID NO: 30 Vl
SSELTQDPAVSVALGQTVRITCQGDSLRSYYASWYQQKPGQAPVLVI
YGKNNRPSGIPDRFSGSSSGNTASLTITGAQAEDEADYYCQHFWSTP
YTFGGGTKLTVL

### Additional V_{L} Regions:

SEQ ID NO: 32 Vl
SSELTQDPAVSVALGQTVTITCQGDSLRSLYASWYQQKPGQAPVLVL
YSKNSRPSGIPDRFSGSSSGNTASLTITGARAEDEADYYCQHFWSTP
YTFGGGTKLTVL
SEQ ID NO: 34 Vl
QSVLTQPPSASGSPGQSVTISCTGTSSDVGAYNYVSWYQQYPGKVPK
LIIYEVTKRPSGVPDRFSGSKSGNTASLTVSGLRAEDEADYYCQHFW
STPYTFGGGTKLTVL
SEQ ID NO:37 VkI
DIQMTQSPSSLSASVGDRVTITCRASQSISRWVAWYQQRPGKAPNLL
IYDASSLKSGVPSRFSGSGSGTEFTLTISSLQPEDIATYYCQQFWGT
PYTFGQGTKLEIK

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, accession numbers, patents, and patent applications cited in this specification are herein incorporated by reference as if each was specifically and individually indicated to be incorporated by reference

1. A method of treating bacteremia or preventing the development of bacteremia in a subject infected with an antibiotic-resistant strain of Pseudomonas aeruginosa, the method comprising administering a therapeutically effective amount of a combination of an anti-PcrV antibody that is an antagonist of the Pseudomonas aeruginosa Type III secretion system; and the antibiotic to which the strain of Pseudomonas aeruginosa is resistant.

2. The method of claim 1, wherein the antibiotic is an aminoglycoside.

3. The method of claim 2, wherein the antibiotic is tobramycin.

4. The method of claim 1, wherein the antibiotic induces the Type III secretion system.

5. The method of claim 1, further comprising a step of determining the level of bacteria in the blood.

6. The method of claim 1, wherein the Pseudomonas aeruginosa strain is resistant to the antibiotic in vivo.

7. A method of treating or preventing an antibiotic-resistant Pseudomonas aeruginosa infection in a subject, the method comprising administering a therapeutically effective amount of a combination of an anti-PcrV antibody that is an antagonist of the Type III secretion system and an antibiotic.

8. The method of claim 7, wherein the antibiotic is ineffective when administered at its maximum tolerated dose in the absence of administration of the antibody.

9. The method of claim 7, wherein administration of the antibiotic and the anti-PcrV antibody does not have increased toxicity to the subject compared to the maximum tolerated dose of the antibiotic when administered alone to the subject.

10. The method of claim 7, wherein the antibiotic induces the expression of the Type III secretion system.

11. The method of claim 10, wherein the antibiotic is a tetracycline, minocycline, eoxycycline, demeclocycline or oxytetracycline.

12. A method of treating a subject with a Pseudomonas aeruginosa lung infection, the method comprising administering an anti-PcrV antibody intravenously, intramuscularly, or subcutaneously, and administering an antibiotic into the lung.

13. The method of claim 12, wherein the antibiotic is administered by insufflation.

14. The method of claim 12, wherein the antibiotic is tobramycin or aztreonam.

15. (canceled)

16. The method of claim 12, wherein administering the anti-PcrV antibody and the antibiotic prevents or treats Pseudomonas aeruginosa bacteremia.

17. A method of treating or preventing bacteremia in a subject infected with Pseudomonas aeruginosa in a tissue other than in the lung, the method comprising administering an anti-PcrV antibody intravenously and an antibiotic.

18. The method of claim 17, wherein the tissue is bladder or urinary tract tissue.

19. A method of enhancing the sensitivity of an antibiotic-resistant Pseudomonas aeruginosa strain to the antibiotic when treating a subject infected with the strain, the method comprising administering to the subject the antibiotic and an anti-PcrV antibody that is an antagonist of the Pseudomonas aeruginosa Type III secretion system.

20. The method of claim 19, wherein the antibiotic is piperacillin.

21. The method of claim 1, wherein the subject has cystic fibrosis, is on a mechanical ventilator, is a neutropenic cancer patient, or is a burn patient.

22. The method of claim 1, wherein the antibody is administered intravenously, intramuscularly, subcutaneously or by insufflation.

23. The method of claim 1, wherein the antibiotic is administered intravenously, intramuscularly, or by insufflation.

24. The method of claim 1, wherein the anti-PcrV antibody competes with Mab166 for binding to PcrV.

25. The method of claim 1, wherein the anti-PcrV antibody comprises a V_{L} region that comprises a CDR3 comprising FWGTP (SEQ ID NO:31), wherein the anti-PcrV antibody selectively binds to PcrV.

26. The method of claim 25, wherein the antibody comprises a V_{H} region that has a CDR3 comprising a sequence NRGDIYYDFTY (SEQ ID NO:38).

27. The method of claim 25, wherein the V_{L} region segment has at least 80% identity to a human germline V-segment.

28. The method of claim 25, wherein the V_{L} region comprises a FR4 that has at least 90% identity to the FR4 region of a human germline Jkappa1, Jkappa2, Jkappa 3, Jkappa4, or Jkappa5 segment or at least 90% identity to the FR4 region of a human germline Jlambda 1, Jlambda2, Jlambda3, or Jlambda7 segment.

29. The method of claim 25, wherein the V_{L} region comprises a FR4 that has at least 90% identity to the FR4 region of the human JK2 germline gene segment or at least 90% identity to the JL2 germline sequence; and a V-segment that has at least 80% identity to a human germline Vkappa I or Vkappa III sequence, or at least 80% identity to a human germline Vlambda sequence.

30. The method of claim 25, wherein the a V_{L} region CDR3 has the sequence Q(Q/H)FWGTPYT (SEQ ID NO:33).

31. The method of claim 1, wherein the anti-PcrV antibody comprises:
a V_{H} region that comprises a CDR3 having a sequence NRGDIYYDFTY (SEQ ID NO:38), a FR4 and a V-segment, wherein the FR4 comprises at least 90% identity to the FR4 region of the human JH3 or human JH6 segment and the V-segment comprises at least 80% identity to the human VH1-18 subclass V-segment or to the human VH3-30.3 V segment.

32. The method of claim 31, wherein the V_{H} region comprises a CDR3 having a sequence NRGDIYYDFTYA(M/F)DX₁ (SEQ ID NO:39), wherein X₁ is I, Q, Y, or S.

33. The method of claim 31, wherein the antibody comprises:
a V_{H} region that comprises a CDR3 having a sequence NRGDIYYDFTYAMDX₁ (SEQ ID NO:40) wherein X₁ is I, Q, Y, or S; a FR4 and a V-segment, wherein the FR4 comprises at least 90% identity to the FR4 region of the human germline JH3 segment or the FR4 region of the human germine JH6 segment, and the V-segment comprises at least 80% identity to the human germline VH1-18 subclass V-segment or to the human germline VH3-30.3 subclass V segment, with the proviso that when X₁ is Y, the FR4 region is not WGQGTSVTVSS (SEQ ID NO:41).

34. The method of claim 33, wherein the antibody comprises:
a V_{H} region that comprises a CDR3 having a sequence NRGDIYYDFTYAMDX₁ (SEQ ID NO:40), wherein X₁ is I, Q, Y, or S; a FR4 and a V-segment, wherein the FR4 comprises at least 90% identity to the FR4 region of the human germline JH3 segment or the FR4 region of the human germline JH6 segment, and the V-segment comprises at least 80% identity to the human germline VH1-18 subclass V-segment or to the human germline VH3-30.3 subclass V segment, with the proviso that when X₁ is Y, the FR4 region is not WGQGTSVTVSS (SEQ ID NO:41); and
a V_{L} region that comprises a CDR3 comprising FW(S/G)TP (SEQ ID NO:42), a FR4 and a V-segment, wherein the FR4 comprises at least 90% identity to the FR4 region of the human germline JK2 gene segment or to the FR4 region of the human germline JL2 segment; and the V-segment comprises at least 80% identity to the human germline VKI L12 sequence, or at least 80% identity to a Vkappa III sequence, or at least 80% identity to a human germline Vlambda2 2c or Vlambda3 31 segment.

35. The method of claim 34, wherein the FR4 of the V_{H} region has the sequence WGQGTX₂VTVSS (SEQ ID NO:43), wherein X₂ is T or M.

36. The method of claim 33, wherein the light chain CDR3 has the sequence Q(H/Q)FW(G/S)TPYT (SEQ ID NO:44).

37. The method of claim 33, wherein the FR4 of the V_{L} region has the sequence FGQGTKLEIK (SEQ ID NO:45) or FGGGTKLTVL (SEQ ID NO:46).

38. The method of claim 31, wherein the anti-PcrV antibody comprises a V_{H} region V-segment has at least 80% identity to the human germline VH3-30.3 segment and the heavy chain region CDR1 comprises the sequence X₃X₄X₅X₆H, wherein X₃ is S, T, or N; X₄ is Y or A; X₅ is A, G, or P; and X₆ is M, I, or L; and the heavy chain region CDR2 comprises the sequence X₇IX₈YX₉GX₁₀X₁₁X₁₂X₁₃Y(A/I)X₁₄SVKG (SEQ ID NO:47), wherein X₇ is V, F, or N; X₈ is S or W; X₉ is D or N; X₁₀ is S, K, R or Y; X₁₁ is N, S, D or E; X₁₂ is K, I, or E; X₁₃ is Y, S, D or W; and X₁₄ is D or S.

39. (canceled)

40. The method of claim 38, wherein the CDR1 is TAGMH (SEQ ID NO:48), SYGIH (SEQ ID NO:49), SYGMH (SEQ ID NO:50), SYPLH (SEQ ID NO:51), or NYPMH (SEQ ID NO:52); and/or the CDR2 is VIWYNGKEISYADSVKG (SEQ ID NO:53), FISYDGSEKYYASSVKG (SEQ ID NO:54), VISYDGSEKWYADSVKG (SEQ ID NO:55), VIWYDGRNKYYADSVKG (SEQ ID NO:56), VIWYDGYNKDYADSVKG (SEQ ID NO:57), or NIWYDGSSESYIDSVKG (SEQ ID NO:58).

41. (canceled)

42. The method of claim 31, wherein the V_{H} region V-segment has at least 80% identity to the human germline VH1-18 sub-class V-segment and the CDR1 has the sequence DHAIS (SEQ ID NO:59) and the CDR2 has the sequence WISPYSGNPNYAQSLQG (SEQ ID NO:60).

43. (canceled)

44. The method of claim 1, wherein the anti-PcrV antibody comprises:
a V_{H} region that has a CDR3 sequence NRGDIYYDFTYAFDI (SEQ ID NO:61), a CDR1 sequence DHAIS (SEQ ID NO:59) and a CDR2 sequence WISPYSGNPNYAQSLQG (SEQ ID NO:60).

45. The method of claim 44, wherein the V-segment of the V_{H} region comprises at least 80% identity to the human germline VH1-18 subclass V-segment.

46. The method of claim 31, wherein the V_{H} region comprises the V-segment region of an amino acid sequence selected from the group consisting of SEQ ID NOs 1, 3, 5, 7, 9, 11, 13, 15, 17, 19, 21, 23, 25, 26, 27, 29, and 35.

47. The method of claim 46, wherein the V_{H} region comprises an amino acid sequence selected from the group consisting of SEQ ID NOs 1, 3, 5, 7, 9, 11, 13, 15, 17, 19, 21, 23, 25, 26, 27, 29, and 35.

48. The method of claim 25, wherein the V_{L} region V-segment comprises at least 90% identity to a human germline Vkappa 1 L12 or Vkappa III sequence, or at least 90% identity to a human germline Vlambda3 31 or to a Vlambda2 2c sequence.

49. The method of claim 48, wherein the V_{L} region V-segment has at least 80% amino acid sequence identity to the human germline VKI L12 segment and the CDR1 has the sequence RASX₁₅X₁₆X₁₇X₁₈X₁₉X₂₀X₂₁A (SEQ ID NO:62), where X₁₅ is Q or E; X₁₆ is S or G; X₁₇ is I or V; X₁₈ is S or D; X₁₉ is S, R, or T; X₂₀₁S W or Y; and X₂₁ is L or V; and the CDR2 has the sequence X₂₁ASX₂₂LX₂₃S (SEQ ID NO:63), wherein X₂₁ is D or A; X₂₂ is S, A, or T; and X₂₃ is E, Q, or K.

50. (canceled)

51. The method of claim 49, wherein the CDR1 has the sequence RASQGISTYLA (SEQ ID NO:64), RASQGISSWLA (SEQ ID NO:65), RASQSISRWVA (SEQ ID NO:66), or RASEGVDRWLA (SEQ ED NO:67); and/or the CDR2 has the sequence AASSLQS (SEQ ID NO:68), DASSLKS (SEQ ID NO:69), DASALQS (SEQ ID NO:70), or DASTLQS (SEQ ID NO:71).

52. (canceled)

53. The method of claim 25, wherein the V_{L} region V-segment has at least 80% amino acid sequence identity to the human germline VKIII L2 sequence and the CDR1 has the sequence RASNSVGAYNLA (SEQ ID NO:72) or RASQSVSSNLA (SEQ ID NO:73); and the CDR2 has the sequence (A/G)AS(T/R)RA(T/P) (SEQ ID NO:74).

54. (canceled)

55. The method of claim 25, wherein the V_{L} region V-segment has at least 80% amino acid sequence identity to a human germline Vlambda3 31 segment and the CDR1 has the sequence QGDSLRS(Y/L)YAS (SEQ ID NO:75); and the CDR2 has the sequence (G/S)KN(N/S)RPS (SEQ ID NO:76).

56. (canceled)

57. The method of claim 25, wherein the V_{L} region V-segment has at least 80% amino acid sequence identity to a human germline Vlambda2 2c segment and the CDR1 has the sequence TGTSSDVGAYNYVS (SEQ ID NO:77) or TGTSSDYVS (SEQ ID NO:78); and the CDR2 has the sequence (E/D)VT(KIN)RPS (SEQ ID NO:79).

58. (canceled)

59. The method of claim 48, wherein the V_{L} region comprises the V-segment of an amino acid sequence selected from the group consisting of SEQ ID NO:2, 4, 6, 8, 10, 12, 14, 16, 18, 20, 22, 24, 28, 30, 32, 34, 36, and 37.

60. The method of claim 59, wherein the V_{L} region comprises an amino acid sequence selected from the group consisting of SEQ ID NO:2, 4, 6, 8, 10, 12, 14, 16, 18, 20, 22, 24, 28, 30, 32, 34, 36, and 37.

61. The method of claim 1, wherein the V_{H} region of the anti-PcrV antibody comprises an amino acid sequence selected from the group consisting of SEQ ID NOs 1, 3, 5, 7, 9, 11, 13, 15, 17, 19, 21, 23, 25, 26, 27, 29, and 35; and the V_{L} region of the anti-PcrV antibody comprises an amino acid sequence selected from the group consisting of SEQ ID NO:2, 4, 6, 8, 10, 12, 14, 16, 18, 20, 22, 24, 28, 30, 32, 34, 36, and 37.

62. The method of claim 1, wherein the anti-PcrV antibody is a Fab′ fragment or an IgG.

63. (canceled)

64. The method of claim 1, wherein the antibody is PEGylated.

65. The method of claim 64, wherein the antibody is di-PEGylated.

66. The method of claim 1, wherein the V_{H} region or the V_{L} region, or both the V_{H} and V_{L} region amino acid sequences comprise a methionine at the N-terminus.
